(19) United States
(12) Patent Application Publication
(10) Pub. No.: US 2006/0030608 A1
(43) Pub. Date: Feb. 9, 2006

(54) Anti aromatase compounds pharmaceutical compositions and uses thereof

(75) Inventors: Richard More Nelson, Newtown, CT (US); Pingrong Liu, Southbury, CT (US); John Robert Proudfoot, Newtown, CT (US); Doris Riether, New York, NY (US); Christian Hanke Justus Joachim Harcken, New Milford, CT (US); David S. Thomson, Ridgefield, CT (US)

Correspondence Address: MICHAEL P. MORRIS;BOEHRINGER INGELHEIM CORPORATION, 900 RIDGEBURY ROAD, P O BOX 368, RIDGEFIELD, CT, 06877-0368, US

(73) Assignee: Boehringer Ingelheim Pharmaceuticals, Inc., Ridgefield, CT (US)

(21) Appl. No.: 11/137,281

(22) Filed: May 25, 2005

Related U.S. Application Data
(60) Provisional application No. 60/598,612, filed on Aug. 4, 2004.

(51) Int. Cl. A61K 31/424; A61K 31/42; A61K 31/416; A61K 31/4184

(52) U.S. Cl. 514/375; 514/379; 514/394; 514/406; 548/217; 548/241; 548/305.4; 548/361.1

(57) ABSTRACT

Compounds of Formula (I) 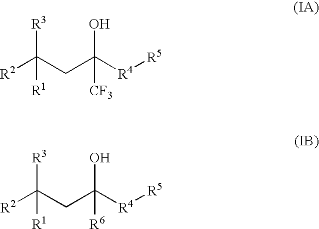
wherein R¹, R², R³, R⁴, R⁵, and R⁶ are as defined herein for Formula (IA) and Formula (IB), or a tautomer, prodrug, solvate, or salt thereof, pharmaceutical compositions containing such compounds, and methods of modulating estrogen receptor activity in a cell or patient or treating an estrogen receptor-mediated disorder, particularly breast and other cancers, in a patient in need thereof by administering an effective amount of compound of the invention thereto.

### RELATED APPLICATIONS

This application claims benefit of U.S. Ser. No. 60/598,612, filed Aug. 4, 2004, which is hereby incorporated by reference in its entirety.

### FIELD OF THE INVENTION

The present invention relates to anti-aromatase compounds, their use in pharmaceutical compositions, and their use in inhibiting the enzyme aromatase for treating disease states or conditions, mediated by the enzyme aromatase, in a patient in need of such a treatment, and other uses.

Aromatase (EC 1.14.14.1), also called estrogen synthetase, is a cytochrome P450 enzyme which catalyzes the formation of aromatic C18 estrogens from C19 androgens; it is symbolized CYP19. Aromatase is located in the ovary and placenta and participates in the regulation of reproductive functions. The enzyme is also widely distributed in extragonadal tissues such as muscle, liver, hair follicles, adipose tissue, bone, and brain.

Mechanistic studies have been undertaken to elucidate the mechanism of aromatase conversion of androgens to estrogens. M. Akhtar et al., J. Steroid Biochem. Mol. Biol., 1993, 44, pp. 375-387; S. Graham-Lorence et al., Protein Science, 1995, 4, pp. 1065-1080; K. R. Korzekwa et al., Biochemistry, 1991, 30, 6155-6162; K. R. Korzekwa et al., J. Steroid Biochem. Mol. Biol., 1993, 44, pp. 367-373.

Breast tumors can either be sensitive to estrogen/estrogen-receptor positive (ER+), sensitive to progesterone/progesterone-receptor positive (PR+), sensitive to both (ER+ and PR+), or neither (ER− and PR−). “Receptor status” is an important part of an invasive breast cancer diagnosis, and is usually provided in the tumor pathology report.

More than two-thirds of breast cancers are considered “estrogen sensitive” because they use estrogen to grow. Aromatase inhibitors reduce the amount of circulating estrogen in postmenopausal women, causing these estrogen-sensitive (ER+ and/or PR+) tumors, which have receptors on their surface that can attach to estrogen, to stop growing or shrink. Thus, if a woman's cancer is determined to be ER+ or PR+, hormonal therapy is a common, and often effective, treatment approach.

Aromatase inhibitors are a family of hormonal treatments that have shown significant activity against breast cancer in postmenopausal women with estrogen-sensitive tumors. Currently, there are three FDA-approved aromatase inhibitors for breast cancer: ARIMIDEX® (anastrazole), disclosed in U.S. Pat. No. 4,935,437; AROMASIN® (exemestane), disclosed in U.S. Pat. No. 4,808,616; and FEMARA® (letrozole), disclosed in U.S. Pat. No. 4,978,672.

ARIMIDEX® (anastrozole), FEMARA® (letrozole), and AROMASIN® (exemestane) are primarily intended for treatment of postmenopausal women with metastatic breast cancer (cancer that has spread beyond the breast). Each is taken in pill form. In the past, these medications were most commonly used by women who may have already tried other antiestrogen therapies, such as tamoxifen, and whose cancer was no longer controlled by those drugs. With the results of new studies, now many doctors recommend an aromatase inhibitor before tamoxifen for postmenopausal women with metastatic disease. In addition, one ongoing study offers the aromatase inhibitor ARIMIDEX® to postmenopausal women with earlier-stage disease after completing five years of tamoxifen. When the results of these studies become known, standard treatment recommendations may change.

ARIMIDEX®, FEMARA®, and AROMASIN® have slightly different approved uses (indications). ARIMIDEX® is approved for adjuvant treatment (treatment following surgery with or without radiation) of postmenopausal women with hormone receptor-positive early breast cancer; ARIMIDEX® is also indicated for first-line treatment (first hormonal treatment in advanced breast cancer) for postmenopausal women with hormone receptor-positive or hormone receptor-unknown locally advanced or metastatic breast cancer; and ARIMIDEX® is also indicated for treatment of advanced breast cancer in postmenopausal women with disease progression following tamoxifen therapy (patients with estrogen receptor-negative disease and patients who did not respond to previous tamoxifen therapy rarely responded to ARIMIDEX®). FEMARA® is approved for first-line treatment of postmenopausal women with hormone receptor positive or hormone receptor unknown locally advanced or metastatic breast cancer or advanced breast cancer in postmenopausal women with disease progression following antiestrogen therapy. AROMASIN®, which is an irreversible steroidal aromatase inhibitor, is indicated for the treatment of advanced breast cancer in postmenopausal women whose disease has progressed following tamoxifen therapy.

Tamoxifen, one of the most commonly used hormonal treatment, blocks estrogen from getting to the estrogen receptor and is useful in both premenopausal and postmenopausal women. In contrast, aromatase inhibitors reduce estrogen levels by preventing aromatase from converting androgens into estrogen. Although pre- and postmenopausal women can use tamoxifen as hormonal therapy, only postmenopausal women should use aromatase inhibitors because postmenopausal women get most of their estrogen from the conversion of androgens into estrogen by the aromatase enzyme route, while premenopausal women get most of their estrogen directly from their ovaries. Thus, tamoxifen therapy functionally mimics the removal of the ovaries (oophorectomy) in premenopausal women while antiaromatase therapy functionally mimics the removal of the adrenal glands (adrenalectomy) of postmenopausal women that was the standard surgical treatment for breast cancer prior to development of effective drug therapies.

Nonetheless, ongoing studies combining aromatase inhibitors with other agents suggest that there may be a role for these drugs in premenopausal women. For example, by combining aromatase inhibitors with luteinizing hormone-releasing hormone analogues allows the possibility of treating premenopausal women with ER+ breast cancer or benign conditions, such as cyclical breast pain, fibroadenomata, recurrent cystic disease, or endometriosis

Several estrogen-dependent diseases exist which could be treated with aromatase inhibitors. These include breast cancer, endometriosis, polycystic ovarian disease, benign breast disease, and endometrial cancer. A beneficial effect of antiestrogens in the treatment of breast cancer has been well established (see Br. J. Cancer, 1971, 25, 270).

Endometriosis is characterized by an abnormal proliferation of the endometrium of the uterus. Since the endometrium is dependent on estradiol for its growth, an inhibitor of estrogen production should stop the progression of the disease.

Benign breast disease, often called fibrocystic breast disease, appears to be dependent on ovarian steroids. See Cancer, 49, 2534 (1982). Aromatase inhibitors have not been tried in this disease, but antiestrogens seem to be of benefit. See Obstet. Gynecol., 54, 80 (1979).

Polycystic ovarian disease is one of the most common causes of infertility in women. The disease appears to result from an abnormality in steroid metabolism, and the major form of therapy in this disease is the antiestrogen clomiphene. See Clin. Endocrinol., 12, 177 (1980).

There are also a variety of hormone-dependent conditions where endocrine therapy may prove beneficial in men with conditions such as gynaecomastia or prostrate cancer. See Aromatase Inhibition and Breast Cancer, Marcel Dekker Inc., New York (2001) 259-266 and 271-278.

Estrogens are synthesized from androgenic steroids. In the biosynthetic pathway for estrogen formation, aromatization is an essential step. It is generally believed that a useful treatment for estrogen-dependent disorders could be obtained if the aromatase enzyme could be effectively inhibited (see Cancer Research, 1982, 42, Suppl. 8: 3261s).

The non-steroidal aromatase inhibitors anastrazole and letrozole have also recently been approved as a first-line endocrine treatment for postmenopausal women with ER+ metastatic breast cancer. Estrogen deprivation by aromatase inhibitors in postmenopausal women, however, may have detrimental effects on bone (See Review in Expert Opin. Pharmacother. 2004, 5(2): 307-316 and references cited therein). Hence compounds of this invention may be useful in combination with agents used for treating osteoporosis. Furthermore, it may be possible to develop selective aromatase modulators that inhibit aromatase in an organ such as the breast, but allow estrogen synthesis in other parts such as the bone.

Besides the above FDA-approved non-steroidal aromatase inhibitors, novel aromatase inhibitors have been described in the scientific and patent literature. For example, U.S. Pat. No. 5,559,141 describes non-steroidal imidazole derivatives that exhibit aromatase and desmolase inhibiting properties and antitumor activity. U.S. Pat. No. 5,587,392 describes novel azolyl methyl phenyl derivatives that inhibit aromatase with higher specificity and greater safety and can be potentially used for the treatment or prevention of a variety of estrogen dependent diseases. European Patent No. 755931 discloses 4-(heterocyclylphenyl)-1,4-dihydropyridines and 4-(heterocyclylphenyl)pyridines as aromatase inhibitors for potential treatment of a variety of estrogen-dependent disorders. PCT International Publication No. WO 98/18791 describes non-steroidal heterocyclic furan compounds as aromatase inhibitors for the treatment and prevention of cancer of the breast, ovary, uterus, pancreas and endometrium as well as for the prevention of benign prostate hypertrophy. PCT International Publication No. WO 02/1990143 discloses trifluoromethyl-substituted alcohols, however, these are potential anti-inflammatory compounds that bind to the glucocorticoid receptor.

### SUMMARY OF THE INVENTION

The instant invention is directed to compounds of Formula (IA) 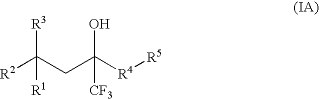
wherein:
R¹ is an aryl or heteroaryl group, each optionally independently substituted with one to three substituent groups,
   wherein each substituent group of R¹ is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, C₁-C₅ alkanoyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, or amino wherein the nitrogen atom is optionally independently mono- or di-substituted by C₁-C₅ alkyl or aryl; or ureido wherein either nitrogen atom is optionally independently substituted with C₁-C₅ alkyl; or C₁-C₅ alkylthio wherein the sulfur atom is optionally oxidized to a sulfoxide or sulfone,
      wherein each substituent group of R¹ is optionally independently substituted with one to three substituent groups selected from methyl, methoxy, halogen, hydroxy, oxo, cyano, or amino,
R² and R³ are each independently hydrogen or C₁-C₅ alkyl;
R⁴ is C₁-C₅ alkyl optionally independently substituted with one to three substituent groups,
   wherein each substituent group of R⁴ is independently C₁-C₃ alkyl, hydroxy, halogen, amino, or oxo; and
R⁵ is a heteroaryl group optionally independently substituted with one to three substituent groups,
   wherein each substituent group of R⁵ is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, or amino wherein the nitrogen atom is optionally independently mono- or di-substituted by C₁-C₅ alkyl; or ureido wherein either nitrogen atom is optionally independently substituted with C₁-C₅ alkyl; or C₁-C₅ alkylthio wherein the sulfur atom is optionally oxidized to a sulfoxide or sulfone,
      wherein each substituent group of R⁵ is optionally independently substituted with one to three substituent groups selected from C₁-C₃ alkyl, C₁-C₃ alkoxy, halogen, hydroxy, oxo, cyano, amino, or trifluoromethyl,
         or a tautomer, prodrug, solvate, or salt thereof.

Another aspect of the invention includes compounds of Formula (IA), wherein:
R¹ is phenyl, naphthyl, dihydrobenzofuranyl, benzofuranyl, chromanyl, dihydrobenzothienyl, benzothienyl, benzodioxolanyl, dihydrobenzoxazolyl, benzoxazolyl, benzisoxazolyl, benzpyrazolyl, or benzimidazolyl, each optionally independently substituted with one to three substituent groups,
   wherein each substituent group of R¹ is independently C₁-C₃ alkyl, C₂-C₃ alkenyl, C₂-C₃ alkynyl, C₁-C₃ alkoxy, C₂-C₃ alkenyloxy, C₁-C₃ alkanoyl, C₁-C₃ alkoxycarbonyl, C₁-C₃ alkanoyloxy, halogen, hydroxy, carboxy, cyano, trifluoromethyl, trifluoromethoxy, nitro, or C₁-C₃ alkylthio wherein the sulfur atom is optionally oxidized to a sulfoxide or sulfone,
      wherein each substituent group of R¹ is optionally independently substituted with a substituent group selected from methyl, methoxy, halogen, hydroxy, oxo, cyano, or amino;
R² and R³ are each independently hydrogen or C₁-C₃ alkyl;
R⁴ is CH₂; and
R⁵ is an indolyl, azaindolyl, diazaindolyl, furanopyridinyl, furanopyrimidinyl, thienopyridinyl, thienopyrimidinyl, oxazolopyridinyl, thiazolopyridinyl, benzimidazolyl, imidazolopyridinyl, quinolinyl, or isoquinolinyl group, each optionally independently substituted with one to three substituent groups,
   wherein each substituent group of R⁵ is independently C₁-C₃ alkyl, phenyl, C₁-C₃ alkoxy, methoxycarbonyl, aminocarbonyl, C₁-C₃ alkylaminocarbonyl, C₁-C₃ dialkylaminocarbonyl, heterocyclylcarbonyl, fluoro, chloro, bromo, cyano, trifluoromethyl, or C₁-C₃ alkylthio wherein the sulfur atom is optionally oxidized to a sulfoxide or sulfone,
      wherein each substituent group of R⁵ is optionally independently substituted with a substituent group selected from methyl, methoxy, fluoro, chloro, bromo, oxo, trifluoromethyl, or amino wherein the nitrogen atom is optionally independently mono- or di-substituted by C₁-C₅ alkyl or aryl;
         or a tautomer, prodrug, solvate, or salt thereof.

The following are representative compounds of Formula (IA) according to the invention:

| | |
|---|---|
| 1,1,1-Trifluoro-4-(5-fluoro-2- methoxyphenyl)-4-methyl-2-(1H- pyrrolo[2,3-c]pyridin-2-ylmethyl)pentan-2- ol | 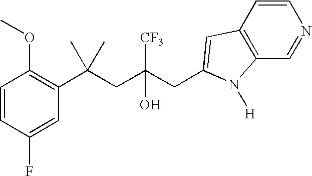 |
| 4-Fluoro-2-[4,4,4-trifluoro-3-hydroxy-1,1- dimethyl-3-(1H-pyrrolo[2,3-c]pyridin-2- ylmethyl)butyl]phenol | 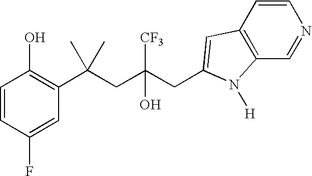 |
| 1,1,1-Trifluoro-4-(5-fluoro-2- methoxyphenyl)-4-methyl-2-(1H- pyrrolo[3,2-c]pyridin-2-ylmethyl)pentan-2- ol | 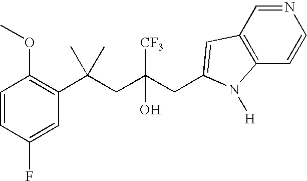 |
| 4-Fluoro-2-[4,4,4-trifluoro-3-hydroxy-1,1- dimethyl-3-(1H-pyrrolo[3,2-c]pyridin-2- ylmethyl)butyl]phenol | 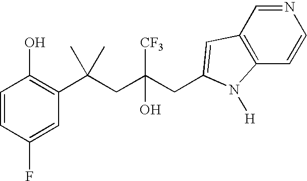 |
| 1,1,1-Trifluoro-4-(3-fluorophenyl)-4- methyl-2-(1H-pyrrolo[2,3-c]pyridin-2- ylmethyl)pentan-2-ol | 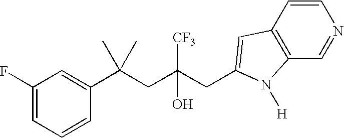 |
| 1,1,1-Trifluoro-4-(4-fluorophenyl)-4- methyl-2-(1H-pyrrolo[2,3-c]pyridin-2- ylmethyl)pentan-2-ol | 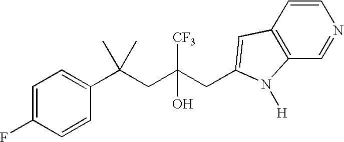 |
| 4-(2,3-Dihydrobenzofuran-7-yl)-1,1,1- trifluoro-4-methyl-2-(1H-pyrrolo[2,3- c]pyridin-2-ylmethyl)pentan-2-ol | 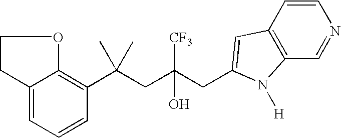 |
| 1,1,1-Trifluoro-4-methyl-4-phenyl-2-(1H- pyrrolo[2,3-c]pyridin-2-ylmethyl)pentan-2- ol | 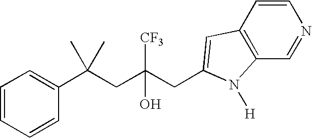 |
| 1,1,1-Trifluoro-4-(4-fluoro-2- methoxyphenyl)-4-methyl-2-(1H- pyrrolo[2,3-c]pyridin-2-ylmethyl)pentan-2- ol | 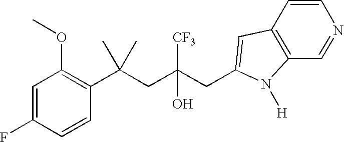 |
| 1,1,1-Trifluoro-4-methyl-4-phenyl-2-(1H- pyrrolo[3,2-c]pyridin-2-ylmethyl)pentan-2- ol | 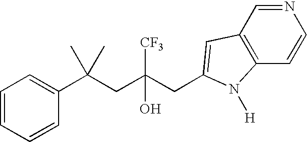 |
| 1,1,1-Trifluoro-4-(4-fluorophenyl)-4- methyl-2-(1H-pyrrolo[3,2-c]pyridin-2- ylmethyl)pentan-2-ol | 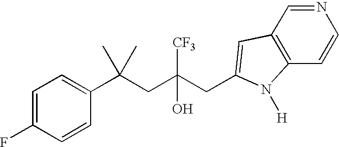 |
| 4-(2,3-Dihydrobenzofuran-7-yl)-1,1,1- trifluoro-4-methyl-2-(1H-pyrrolo[3,2- c]pyridin-2-ylmethyl)pentan-2-ol | 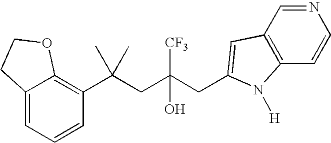 |
| 1,1,1-Trifluoro-4-(4-fluoro-2- methoxyphenyl)-4-methyl-2-(1H- pyrrolo[3,2-c]pyridin-2-ylmethyl)pentan-2- ol | 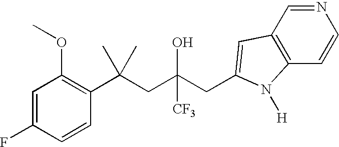 |
| 5-Fluoro-2-[4,4,4-trifluoro-3-hydroxy-1,1- dimethyl-3-(1H-pyrrolo[2,3-c]pyridin-2- ylmethyl)butyl]phenol | 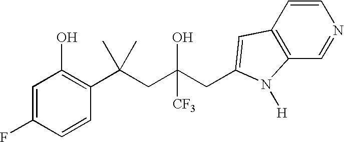 |
| 1,1,1-Trifluoro-4-(5-fluoro-2- methylphenyl)-4-methyl-2-(1H-pyrrolo[2,3- c]pyridin-2-ylmethyl)pentan-2-ol | 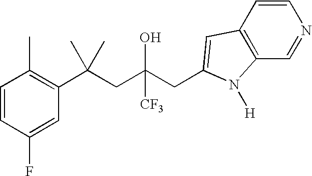 |
| 4-Fluoro-2-[4,4,4-trifluoro-3-hydroxy-1,1- dimethyl-3-(3-methyl-1H-pyrrolo[2,3- c]pyridin-2-ylmethyl)butyl]phenol | 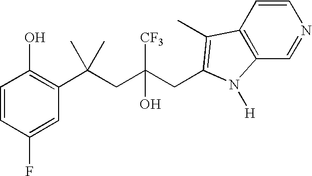 |
| 5-Fluoro-2-[4,4,4-trifluoro-3-hydroxy-1,1- dimethyl-3-(1H-pyrrolo[3,2-c]pyridin-2- ylmethyl)butyl]phenol | 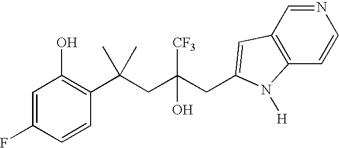 |
| 1,1,1-Trifluoro-4-(3-fluorophenyl)-4- methyl-2-(1H-pyrrolo[3,2-c]pyridin-2- ylmethyl)pentan-2-ol | 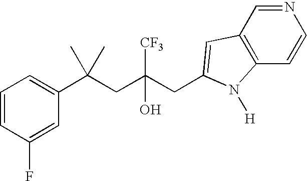 |
| 1,1,1-Trifluoro-4-(5-fluoro-2,3- dihydrobenzofuran-7-yl)-4-methyl-2-(1H- pyrrolo[2,3-c]pyridin-2-ylmethyl)pentan-2- ol | 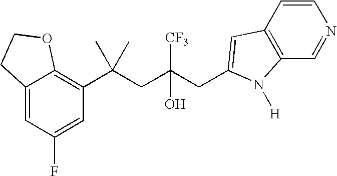 |
| 4-Fluoro-2-[4,4,4-trifluoro-3-hydroxy-1,1- dimethyl-3-(1H-pyrrolo[2,3-c]-[3-methyl- pyridin]-2-ylmethyl)butyl]phenol | 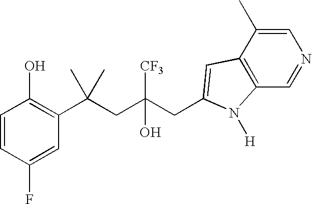 |
| 1,1,1-Trifluoro-4-(5-fluoro-2- methoxyphenyl)-4-methyl-2-(3-methyl-1H- pyrrolo[3,2-c]pyridin-2-ylmethyl)pentan-2- ol | 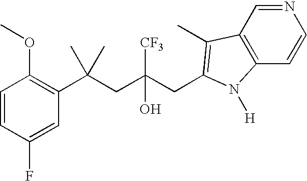 |
| 1,1,1-Trifluoro-4-(5-fluoro-2,3- dihydrobenzofuran-7-yl)-4-methyl-2-(1H- pyrrolo[3,2-c]pyridin-2-ylmethyl)pentan-2- ol | 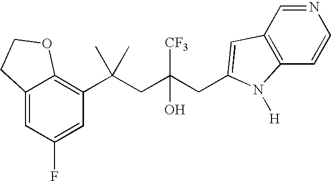 |
| 1,1,1-Trifluoro-4-(5-fluoro-2- methylphenyl)-4-methyl-2-(3-methyl-1H- pyrrolo[2,3-c]pyridin-2-ylmethyl)pentan-2- ol | 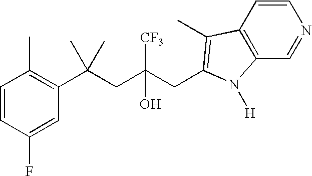 |
| 1,1,1-Trifluoro-4-(5-fluoro-2- methoxyphenyl)-4-methyl-2-(6-methyl-1H- pyrrolo[3,2-c]pyridin-2-ylmethyl)pentan-2- ol | 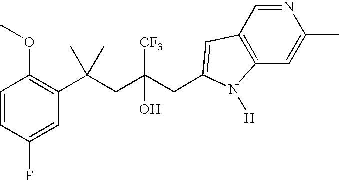 |
| 1,1,1-Trifluoro-4-(5-fluoro-2- methoxyphenyl)-4-methyl-2-(5H- pyrrolo[3,2-d]pyrimidin-6-ylmethyl)pentan- 2-ol | 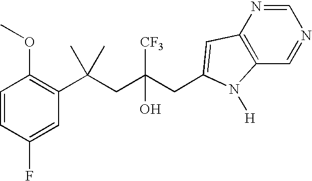 |
| 1,1,1-Trifluoro-4-(5-fluoro-2- methoxyphenyl)-4-methyl-2-(5H- pyrrolo[3,2-c]pyridazin-6-ylmethyl)pentan- 2-ol | 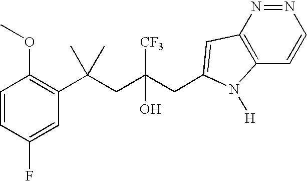 |
| 1,1,1-Trifluoro-4-(5-fluoro-2- methylphenyl)-4-methyl-2-(1H-pyrrolo[2,3- d]pyridazin-2-ylmethyl)pentan-2-ol | 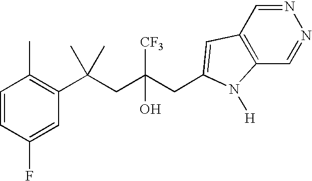 |
| 4-(5-Chloro-2,3-dihydrobenzofuran-7-yl)- 1,1,1-trifluoro-4-methyl-2-(6-methyl-1H- pyrrolo[3,2-c]pyridin-2-ylmethyl)pentan-2- ol | 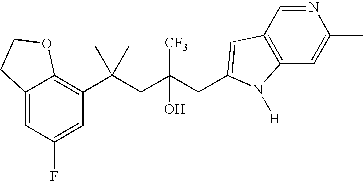 |
| 1,1,1-Trifluoro-4-(5-fluoro-2- methylphenyl)-4-methyl-2-(6-methyl-1H- pyrrolo[3,2-c]pyridin-2-ylmethyl)pentan-2- ol | 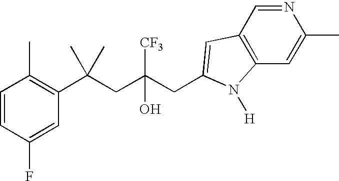 |
| 2-[4-(5-Fluoro-2-methylphenyl)-2-hydroxy- 4-methyl-2-trifluoromethylpentyl]-1H- pyrrolo[3,2-b]pyridine-5-carbonitrile | 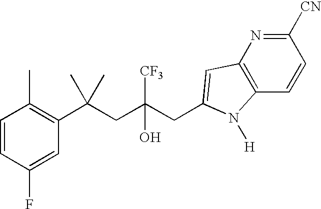 |
| 2-[4-(5-Chloro-2,3-dihydrobenzofuran-7- yl)-2-hydroxy-4-methyl-2- trifluoromethylpentyl]-1H-pyrrolo[3,2- b]pyridine-5-carbonitrile | 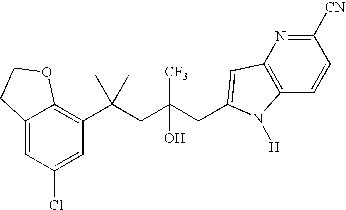 |
| 1,1,1-Trifluoro-4-(5-fluoro-2- methylphenyl)-4-methyl-2-(5H-pyrrolo[3,2- d]pyrimidin-6-ylmethyl)pentan-2-ol | 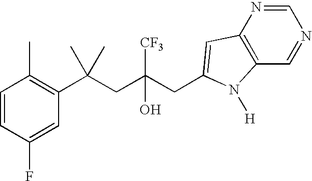 |
| 4-(5-Chloro-2,3-dihydrobenzofuran-7-yl)- 1,1,1-trifluoro-4-methyl-2-(5H-pyrrolo[3,2- d]pyrimidin-6-ylmethyl)pentan-2-ol | 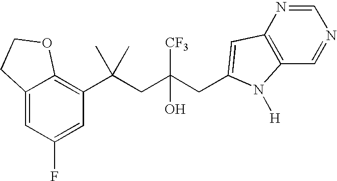 |
| 4-(5-Chloro-2,3-dihydrobenzofuran-7-yl)- 1,1,1-trifluoro-4-methyl-2-(3-methyl-1H- pyrrolo[2,3-c]pyridin-2-ylmethyl)pentan-2- ol | 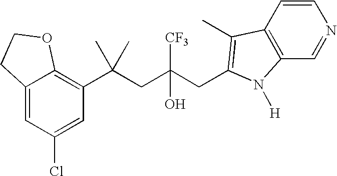 |
| 1,1,1-Trifluoro-4-(5-fluoro-2- methylphenyl)-4-methyl-2-(5H-pyrrolo[3,2- c]pyridazin-6-ylmethyl)pentan-2-ol | 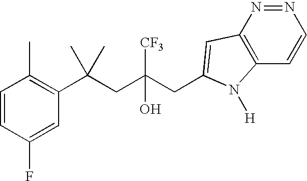 |
| 4-(5-Chloro-2,3-dihydrobenzofuran-7-yl)- 1,1,1-trifluoro-4-methyl-2-(5H-pyrrolo[3,2- c]pyridazin-6-ylmethyl)pentan-2-ol | 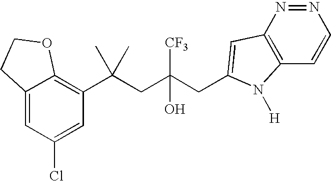 |
| 4-(5-Chloro-2,3-dihydrobenzofuran-7-yl)- 1,1,1-trifluoro-4-methyl-2-(1H-pyrrolo [2,3- d]pyridazin-2-ylmethyl)pentan-2-ol | 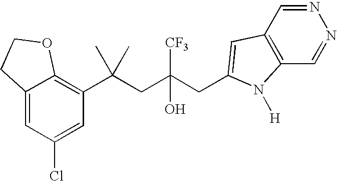 |
| 4-(5-Bromo-2,3-dihydrobenzoftiran-7-yl)- 1,1,1-trifluoro-4-methyl-2-(1H-pyrrolo[2,3- c]pyridin-2-ylmethyl)pentan-2-ol | 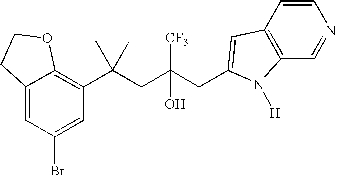 |
| 1,1,1-Trifluoro-4-methyl-4-(5-methyl-2,3- dihydrobenzofuran-7-yl)-2-(1H- pyrrolo[2,3-c]pyridin-2-ylmethyl)pentan-2- ol | 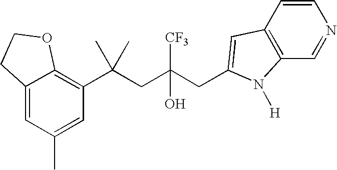 |
| 4-(5-Chloro-2,3-dihydrobenzofuran-7-yl)- 1,1,1-trifluoro-4-methyl-2-(1H-pyrrolo[2,3- c]pyridin-2-ylmethyl)pentan-2-ol | 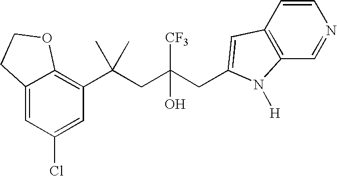 |
| 1,1,1-Trifluoro-4-(5-fluoro-2- methoxyphenyl)-4-methyl-2-(6-oxy-1H- pyrrolo[2,3-c]pyridin-2-ylmethyl)pentan-2- ol | 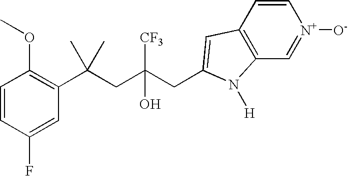 |
| 1,1,1-Trifluoro-4-(5-fluoro-2- methoxyphenyl)-4-methyl-2-thieno[2,3- c]pyridin-2-ylmethylpentan-2-ol | 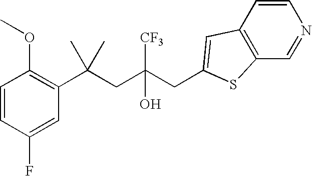 |
| 4-(5-Chloro-2,3-dihydrobenzofuran-7-yl)- 2,4-dimethyl-1-thieno[2,3-c]pyridin-2- ylpentan-2-ol | 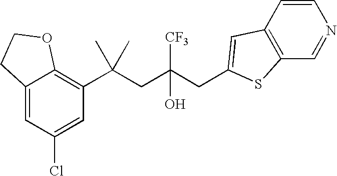 |
| 4-(5-Fluoro-2-methylphenyl)-2,4-dimethyl- 1-thieno[2,3-c]pyridin-2-ylpentan-2-ol | 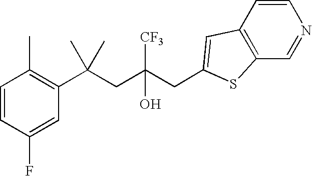 |
| 1,1,1-Trifluoro-4-(5-fluoro-2- methoxyphenyl)-2-furo[2,3-c]pyridin-2- ylmethyl-4-methylpentan-2-ol | 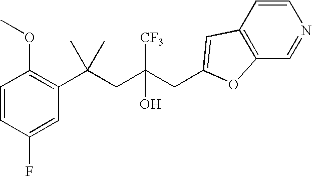 |
| 4-(5-Chloro-2,3-dihydrobenzofuran-7-yl)-1- furo[2,3-c]pyridin-2-yl-2,4-dimethylpentan- 2-ol | 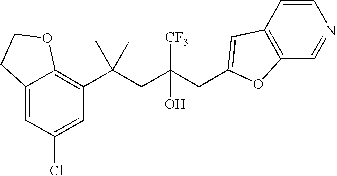 |
| 4-(5-Fluoro-2-methylphenyl)-1-furo[2,3- c]pyridin-2-yl-2,4-dimethylpentan-2-ol | 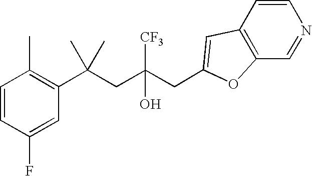 |
| 1,1,1-Trifluoro-4-(5-fluoro-2- methylphenyl)-4-methyl-2-(1H-pyrrolo[3,2- c]pyridin-2-ylmethyl)pentan-2-ol | 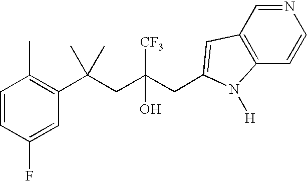 |
| 1,1,1-Trifluoro-4-methyl-4-(5-methyl-2,3- dihydrobenzofuran-7-yl)-2-(1H- pyrrolo[3,2-c]pyridin-2-ylmethyl)pentan-2- ol | 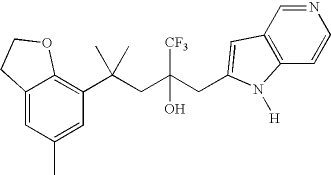 |
| 4-(5-Chloro-2,3-dihydrobenzofuran-7-yl)- 1,1,1-trifluoro-4-methyl-2-(1H-pyrrolo[3,2- c]pyridin-2-ylmethyl)pentan-2-ol | 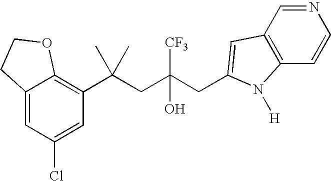 |
| 4-(5-Bromo-2,3-dihydrobenzofuran-7-yl)- 1,1,1-trifluoro-4-methyl-2-(1H-pyrrolo[3,2- c]pyridin-2-ylmethyl)pentan-2-ol | 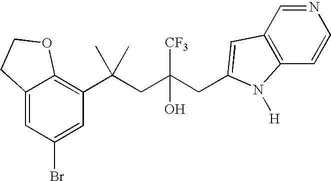 |
| 2-(3-Dimethylaminomethyl-1H- pyrrolo[3,2-c]pyridin-2-ylmethyl)-1,1,1- trifluoro-4-(5-fluoro-2-methoxyphenyl)-4- methylpentan-2-ol | 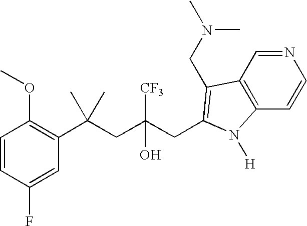 |
| 1,1,1-Trifluoro-4-(5-fluoro-2- methoxyphenyl)-2-furo[3,2-c]pyridin-2- ylmethyl-4-methylpentan-2-ol | 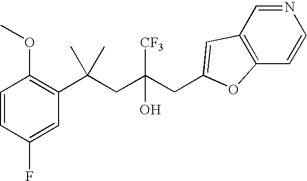 |
| 1,1,1-Trifluoro-4-(5-fluoro-2- methoxyphenyl)-4-methyl-2-thieno[3,2- c]pyridin-2-ylmethylpentan-2-ol | 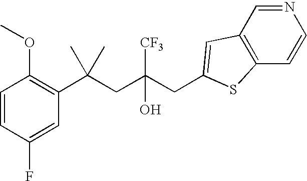 |
| 4-(5-Chloro-2,3-dihydrobenzofuran-7-yl)- 1,1,1-trifluoro-4-methyl-2-thieno[3,2- c]pyridin-2-ylmethylpentan-2-ol | 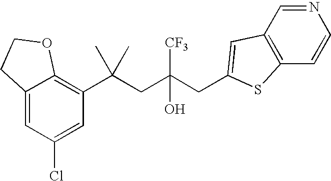 |
| 1,1,1-Trifluoro-4-(5-fluoro-2- methylphenyl)-4-methyl-2-thieno[3,2- c]pyridin-2-ylmethylpentan-2-ol | 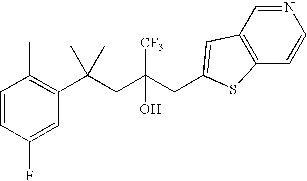 |
| 4-Fluoro-2-(4,4,4-trifluoro-3-hydroxy-1,1- dimethyl-3-thieno[3,2-c]pyridin-2- ylmethylbutyl)phenol | 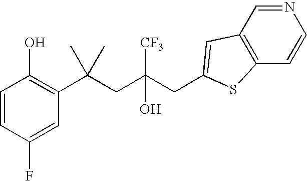 |
| 4-Fluoro-2-(4,4,4-trifluoro-3-furo[3,2- c]pyridin-2-ylmethyl-3-hydroxy-1,1- dimethylbutyl)phenol | 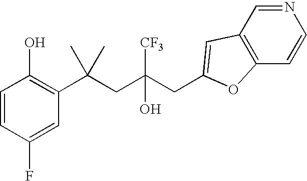 |
| 2-(3-Bromo-1H-indol-2-ylmethyl)-1,1,1- trifluoro-4-(3-fluorophenyl)-4- methylpentan-2-ol | 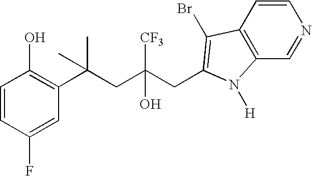 |
| 1,1,1-Trifluoro-4-(5-fluoro-2- methoxyphenyl)-4-methyl-2-(7H- pyrrolo[2,3-d]pyrimidin-6-ylmethyl)pentan- 2-ol | 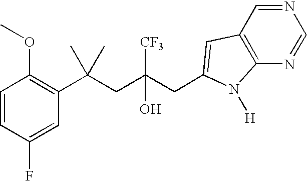 |

or a tautomer, prodrug, solvate, or salt thereof.

Preferred compounds of Formula (IA) include:
1,1,1-Trifluoro-4-(5-fluoro-2-methoxyphenyl)-4-methyl-2-(1H-pyrrolo[2,3-c]pyridin-2-ylmethyl)pentan-2-ol;
1,1,1-Trifluoro-4-(3-fluorophenyl)-4-methyl-2-(1H-pyrrolo[2,3-c]pyridin-2-ylmethyl)pentan-2-ol;
1,1,1-Trifluoro-4-(4-fluorophenyl)-4-methyl-2-(1H-pyrrolo[2,3-c]pyridin-2-ylmethyl)pentan-2-ol;
4-(2,3-Dihydrobenzofuran-7-yl)-1,1,1-trifluoro-4-methyl-2-(1H-pyrrolo[2,3-c]pyridin-2-ylmethyl)pentan-2-ol;
1,1,1-Trifluoro-4-methyl-4-phenyl-2-(1H-pyrrolo[2,3-c]pyridin-2-ylmethyl)pentan-2-ol;
1,1,1-Trifluoro-4-(4-fluoro-2-methoxyphenyl)-4-methyl-2-(1H-pyrrolo[2,3-c]pyridin-2-ylmethyl)pentan-2-ol;
1,1,1-Trifluoro-4-methyl-4-phenyl-2-(1H-pyrrolo [3,2-c]pyridin-2-ylmethyl)pentan-2-ol;
1,1,1-Trifluoro-4-(4-fluorophenyl)-4-methyl-2-(1H-pyrrolo[3,2-c]pyridin-2-ylmethyl)pentan-2-ol;
4-(2,3-Dihydrobenzofuran-7-yl)-1,1,1-trifluoro-4-methyl-2-(1H-pyrrolo[3,2-c]pyridin-2-ylmethyl)pentan-2-ol;
5-Fluoro-2-[4,4,4-trifluoro-3-hydroxy-1,1-dimethyl-3-(1H-pyrrolo[3,2-c]pyridin-2-ylmethyl)butyl]phenol;
1,1,1-Trifluoro-4-(3-fluorophenyl)-4-methyl-2-(1H-pyrrolo[3,2-c]pyridin-2-ylmethyl)pentan-2-ol;
1,1,1-Trifluoro-4-(5-fluoro-2-methoxyphenyl)-4-methyl-2-(5H-pyrrolo[3,2-d]pyrimidin-6-ylmethyl)pentan-2-ol;
4-(5-Chloro-2,3-dihydrobenzofuran-7-yl)-1,1,1-trifluoro-4-methyl-2-(6-methyl-1H-pyrrolo[3,2-c]pyridin-2-ylmethyl)pentan-2-ol;
1,1,1-Trifluoro-4-(5-fluoro-2-methylphenyl)-4-methyl-2-(5H-pyrrolo[3,2-d]pyrimidin-6-ylmethyl)pentan-2-ol;
1,1,1-Trifluoro-4-methyl-4-(5-methyl-2,3-dihydrobenzofuran-7-yl)-2-(1H-pyrrolo[2,3-c]pyridin-2-ylmethyl)pentan-2-ol;
1,1,1-Trifluoro-4-(5-fluoro-2-methoxyphenyl)-4-methyl-2-thieno[2,3-c]pyridin-2-ylmethylpentan-2-ol;
2-(3-Dimethylaminomethyl-1H-pyrrolo[3,2-c]pyridin-2-ylmethyl)-1,1,1-trifluoro-4-(5-fluoro-2-methoxyphenyl)-4-methylpentan-2-ol; and
1,1,1-Trifluoro-4-(5-fluoro-2-methoxyphenyl)-4-methyl-2-(7H-pyrrolo[2,3-d]pyrimidin-6-ylmethyl)pentan-2-ol,
   or a tautomer, prodrug, solvate, or salt thereof.

The instant invention is also directed to compounds of Formula (IB) 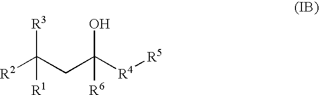
wherein:
R¹ is an aryl or heteroaryl group, each optionally independently substituted with one to three substituent groups,
   wherein each substituent group of R¹ is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, C₁-C₅ alkanoyl, aroyl, aminocarbonyl, C₁-C₅ alkylaminocarbonyl, C₁-C₅ dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, or amino wherein the nitrogen atom is optionally independently mono- or di-substituted by C₁-C₅ alkyl or aryl; or ureido wherein either nitrogen atom is optionally independently substituted with C₁-C₅ alkyl; or C₁-C₅ alkylthio wherein the sulfur atom is optionally oxidized to a sulfoxide or sulfone,
      wherein each substituent group of R¹ is optionally independently substituted with one to three substituent groups selected from methyl, methoxy, halogen, hydroxy, oxo, cyano, or amino,
R² and R³ are each independently C₁-C₅ alkyl;
R⁴ is C₁-C₅ alkyl optionally independently substituted with one to three substituent groups,
   wherein each substituent group of R⁴ is independently C₁-C₃ alkyl, hydroxy, halogen, or oxo;
R⁵ is a heteroaryl group optionally independently substituted with one to three substituent groups,
   wherein each substituent group of R⁵ is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, or amino wherein the nitrogen atom is optionally independently mono- or di-substituted by C₁-C₅ alkyl; or ureido wherein either nitrogen atom is optionally independently substituted with C₁-C₅ alkyl; or C₁-C₅ alkylthio wherein the sulfur atom is optionally oxidized to a sulfoxide or sulfone,
      wherein each substituent group of R⁵ is optionally independently substituted with one to three substituent groups selected from C₁-C₃ alkyl, C₁-C₃ alkoxy, halogen, hydroxy, oxo, cyano, amino, or trifluoromethyl; and
R⁶ is C₁-C₈ alkyl, C₂-C₈ alkenyl, C₂-C₈ alkynyl, carbocycle, heterocyclyl, aryl, heteroaryl, carbocycle-C₁-C₈ alkyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups,
   wherein each substituent group of R⁶ is independently C₁-C₅ alkyl, C₂-C₅ alkenyl, C₂-C₅ alkynyl, C₃-C₈ cycloalkyl, phenyl, C₁-C₅ alkoxy, phenoxy, C₁-C₅ alkanoyl, aroyl, C₁-C₅ alkoxycarbonyl, C₁-C₅ alkanoyloxy, aminocarbonyloxy, C₁-C₅ alkylaminocarbonyloxy, C₁-C₅ dialkylaminocarbonyloxy, aminocarbonyl, C₁-C₅ alkylaminocarbonyl, C₁-C₅ dialkylaminocarbonyl, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, oxo, trifluoromethyl, nitro, amino wherein the nitrogen atom is optionally independently mono- or di-substituted by C₁-C₅ alkyl; or ureido wherein either nitrogen atom is optionally independently substituted with C₁-C₅ alkyl; or C₁-C₅ alkylthio wherein the sulfur atom is optionally oxidized to a sulfoxide or sulfone,
wherein R⁶ cannot be trifluoromethyl,
   or a tautomer, prodrug, solvate, or salt thereof.

Another aspect of the invention includes compounds of Formula (IB), wherein:
R¹ is phenyl, naphthyl, dihydrobenzofuranyl, benzofuranyl, chromanyl, dihydrobenzothienyl, benzothienyl, benzodioxolanyl, benzoxazolyl, benzisoxazolyl, benzpyrazolyl, or benzimidazolyl, each optionally independently substituted with one to three substituent groups,
   wherein each substituent group of R¹ is independently C₁-C₃ alkyl, C₂-C₃ alkenyl, C₂-C₃ alkynyl, C₁-C₃ alkoxy, C₂-C₃ alkenyloxy, C₁-C₃ alkanoyl, C₁-C₃ alkoxycarbonyl, C₁-C₃ alkanoyloxy, halogen, hydroxy, carboxy, cyano, trifluoromethyl, nitro, or C₁-C₃ alkylthio wherein the sulfur atom is optionally oxidized to a sulfoxide or sulfone,
      wherein each substituent group of R¹ is optionally independently substituted with a substituent group selected from methyl, methoxy, halogen, hydroxy, oxo, cyano, or amino;
R² and R³ are each independently C₁-C₃ alkyl;
R⁴ is CH₂;
R⁵ is an indolyl, azaindolyl, diazaindolyl, furanopyridinyl, furanopyrimidinyl, thienopyridinyl, thienopyrimidinyl, oxazolopyridinyl, thiazolopyridinyl, benzimidazolyl, imidazolopyridinyl, quinolinyl, or isoquinolinyl group, each optionally independently substituted with one to three substituent groups,
   wherein each substituent group of R⁵ is independently C₁-C₃ alkyl, phenyl, C₁-C₃ alkoxy, methoxycarbonyl, aminocarbonyl, C₁-C₃ alkylaminocarbonyl, C₁-C₃ dialkylaminocarbonyl, heterocyclylcarbonyl, fluoro, chloro, bromo, cyano, trifluoromethyl, or C₁-C₃ alkylthio wherein the sulfur atom is optionally oxidized to a sulfoxide or sulfone,
      wherein each substituent group of R⁵ is optionally independently substituted with a substituent group selected from methyl, methoxy, fluoro, chloro, bromo, trifluoromethyl, or amino wherein the nitrogen atom is optionally independently mono- or di-substituted by C₁-C₅ alkyl or aryl or trifluoromethyl; and
R⁶ is C₁-C₅ alkyl, C₂-C₅ alkenyl, C₃-C₆ cycloalkyl, phenyl, C₃-C₆ cycloalkyl-C₁-C₃ alkyl, phenyl-C₁-C₃ alkyl, phenyl-C₁-C₃ haloalkyl, C₃-C₆ cycloalkyl-C₂-C₃ alkenyl, phenyl-C₂-C₃ alkenyl, each optionally independently substituted with one to three substituent groups,
   wherein each substituent group of R⁶ is independently C₁-C₃ alkyl, C₂-C₃ alkenyl, C₂-C₃ alkynyl, C₁-C₃ alkoxy, aminocarbonyl, C₁-C₃ alkylaminocarbonyl, C₁-C₃ dialkylaminocarbonyl, halogen, hydroxy, carboxy, cyano, trifluoromethyl, nitro, or C₁-C₃ alkylthio wherein the sulfur atom is optionally oxidized to a sulfoxide or sulfone,
      or a tautomer, prodrug, solvate, or salt thereof.

The following are representative compounds of Formula (IB) according to the invention:

| Compound Name | Compound Structure |
|---|---|
| 2-Cyclopropyl-4-(5-fluoro-2- methoxyphenyl)-4-methyl-1-(1H- pyrrolo[2,3-c]pyridin-2-yl)pentan-2-ol | 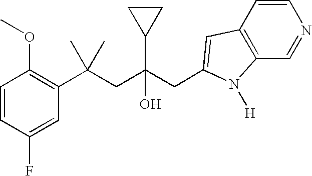 |
| 4-(5-Bromo-2,3-dihydrobenzofuran-7-yl)- 2-cyclopropyl-4-methyl-1-(1H- pyrrolo[2,3-c]pyridin-2-yl)pentan-2-ol | 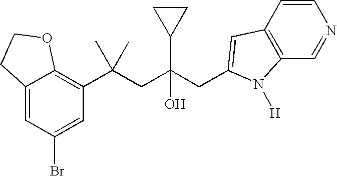 |
| 2-Cyclopropyl-4-(5-fluoro-2- methoxyphenyl)-4-methyl-1-(1H- pyrrolo[3,2-c]pyridin-2-yl)pentan-2-ol | 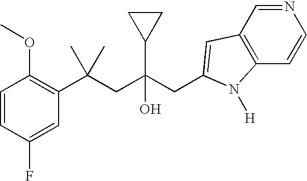 |
| 4-(5-Fluoro-2-methoxyphenyl)-2- hydroxy-4-methyl-2-(1H-pyrrolo[2,3- c]pyridin-2-ylmethyl)pentanoic acid | 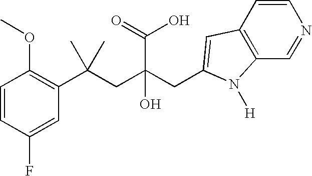 |
| 4-(5-Fluoro-2-methoxyphenyl)-2- hydroxy-4-methyl-2-(1H-pyrrolo[2,3- c]pyridin-2-ylmethyl)pentanoic acid methyl ester | 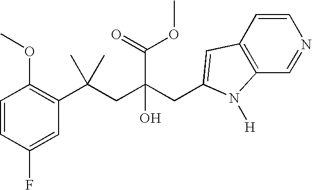 |
| 2-Cyclopropyl-4-(5-fluoro-2- methyiphenyl)-4-methyl-1-(1H- pyrrolo[2,3-c]pyridin-2-yl)pentan-2-ol | 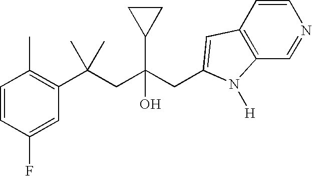 |
| 4-(5-Chloro-2,3-dihydrobenzofuran-7-yl)- 2-cyclopropyl-4-methyl-1-(1H- pyrrolo[2,3-c]pyridin-2-yl)pentan-2-ol | 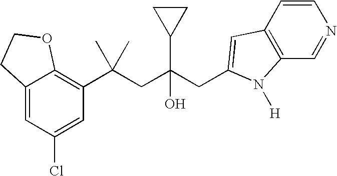 |
| 2-Cyclopropyl-4-(5-fluoro-2- methylphenyl)-4-methyl-1-(1H- pyrrolo[3,2-c]pyridin-2-yl)pentan-2-ol | 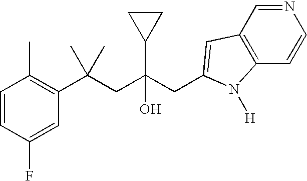 |
| 4-(5-Chloro-2,3-dihydrobenzofuran-7-yl)- 2-cyclopropyl-4-methyl-1-(1H- pyrrolo[3,2-c]pyridin-2-yl)pentan-2-ol | 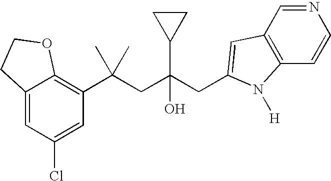 |
| 4-(5-Fluoro-2-methoxyphenyl)-2,4- dimethyl-1-(1H-pyrrolo[2,3-c]pyridin-2- yl)pentan-2-ol | 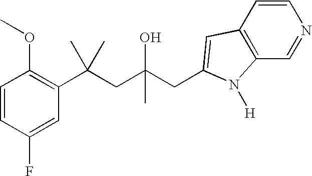 |
| 5-(5-Fluoro-2-methoxyphenyl)-2,5- dimethyl-3-(1H-pyrrolo[2,3-c]pyridin-2- ylmethyl)hexan-3-ol | 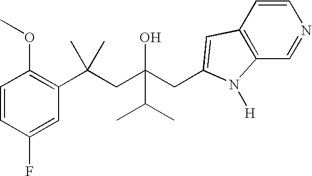 |
| 5-(5-Fluoro-2-methoxyphenyl)-2,2,5- trimethyl-3-(1H-pyrrolo[2,3-c]pyridin-2- ylmethyl)hexan-3-ol | 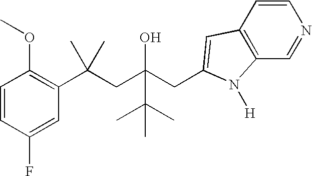 |
| 2-Cyclohexyl-4-(5-fluoro-2- methoxyphenyl)-4-methyl-1-(1H- pyrrolo[2,3-c]pyridin-2-yl)pentan-2-ol | 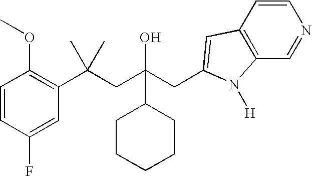 |
| 2-Cyclopentyl-4-(5-fluoro-2- methoxyphenyl)-4-methyl-1-(1H- pyrrolo[2,3-c]pyridin-2-yl)pentan-2-ol | 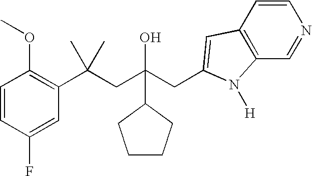 |
| 5-(5-Fluoro-2-methoxyphenyl)-5-methyl- 3-(1H-pyrrolo[2,3-c]pyridin-2- ylmethyl)hexan-3-ol | 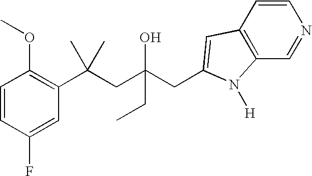 |
| 2-(5-Fluoro-2-methoxyphenyl)-2,6- dimethyl-4-(1H-pyrrolo[2,3-c]pyridin-2- ylmethyl)heptan-4-ol | 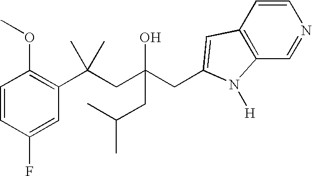 |
| 2-(5-Fluoro-2-methoxyphenyl)-2,5,5- trimethyl-4-(1H-pyrrolo[2,3-c]pyridin-2- ylmethyl)heptan-4-ol | 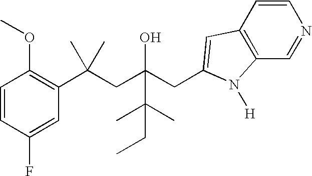 |
| 1,1-Difluoro-4-(5-fluoro-2- methoxyphenyl)-4-methyl-2-(1H- pyrrolo[2,3-c]pyridin-2-ylmethyl)pentan- 2-ol | 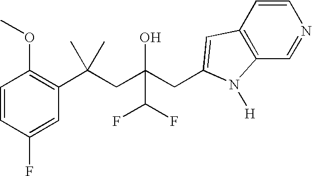 |
| 1-Cyclohexyl-4-(5-fluoro-2- methoxyphenyl)-4-methyl-2-(1H- pyrrolo[2,3-c]pyridin-2-ylmethyl)pentan- 2-ol | 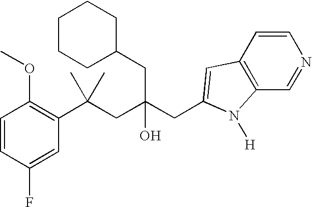 |
| 5-(5-Fluoro-2-methylphenyl)-2,5- dimethyl-3-(1H-pyrrolo[2,3-c]pyridin-2- ylmethyl)hexan-3-ol | 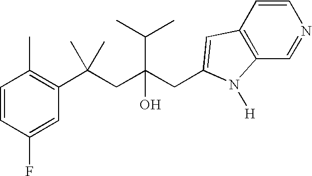 |
| 5-(5-Fluoro-2-methylphenyl)-2,2,5- trimethyl-3-(1H-pyrrolo[2,3-c]pyridin-2- ylmethyl)hexan-3-ol | 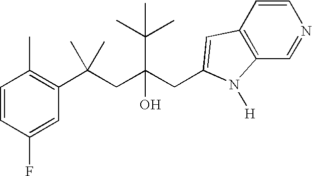 |
| 5-(5-Chloro-2,3-dihydrobenzofuran-7-yl)- 2,5-dimethyl-3-(1H-pyrrolo[2,3-c]pyridin- 2-ylmethyl)hexan-3-ol | 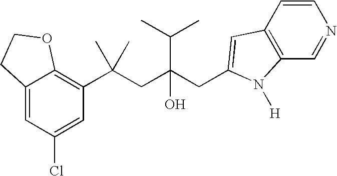 |
| 2-Cyclobutyl-4-(5-fluoro-2- methoxyphenyl)-4-methyl-1-(1H- pyrrolo[2,3-c]pyridin-2-yl)pentan-2-ol | 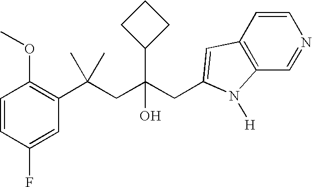 |
| 2-(5-Fluoro-2-methoxyphenyl)-2,6,6- trimethyl-4-(1H-pyrrolo[2,3-c]pyridin-2- ylmethyl)heptan-4-ol | 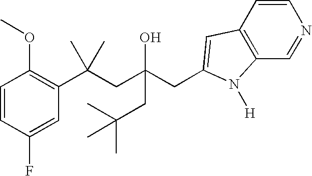 |
| 5-(5-Fluoro-2-methoxyphenyl)-5-methyl- 3-(1H-pyrrolo[2,3-c]pyridin-2- ylmethyl)hex-1-en-3-ol | 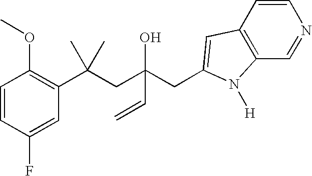 |
| 5-(5-Fluoro-2-methoxyphenyl)-5-methyl- 3-(1H-pyrrolo[2,3-c]pyridin-2- ylmethyl)hex-1-yn-3-ol | 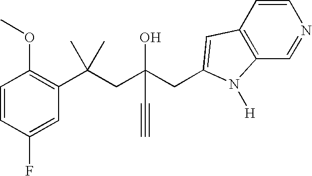 |
| 1-Fluoro-4-(5-fluoro-2-methoxyphenyl)-4- methyl-2-(1H-pyrrolo[2,3-c]pyridin-2- ylmethyl)pentan-2-ol | 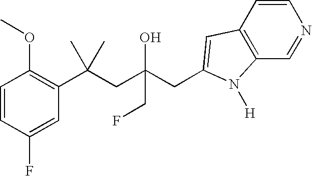 |
| 2,2-Difluoro-5-(5-fluoro-2- methoxyphenyl)-5-methyl-3-(1H- pyrrolo[2,3-c]pyridin-2-ylmethyl)hexan-3- ol | 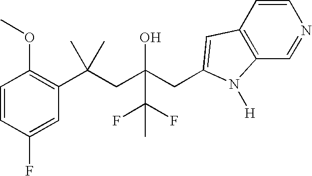 |
| 2-Fluoro-5-(5-fluoro-2-methoxyphenyl)- 2,5-dimethyl-3-(1H-pyrrolo[2,3-c]pyridin- 2-ylmethyl)hexan-3-ol | 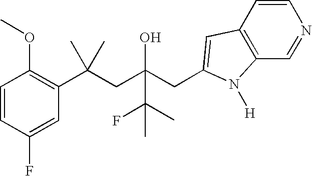 |
| 2-Fluoro-5-(5-fluoro-2-methoxyphenyl)-5- methyl-3-(1H-pyrrolo[2,3-c]pyridin-2- ylmethyl)hexan-3-ol | 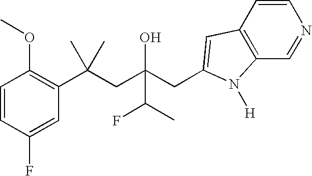 |
| 5-(5-Fluoro-2-methoxyphenyl)-2,5- dimethyl-3-(1H-pyrrolo[2,3-c]pyridin-2- ylmethyl)hex-1-en-3-ol | 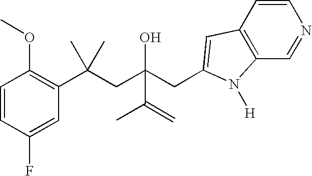 |
| 1,1,1-Trifluoro-5-(5-fluoro-2- methoxyphenyl)-5-methyl-3-(1H- pyrrolo[2,3-c]pyridin-2-ylmethyl)hexan-3- ol | 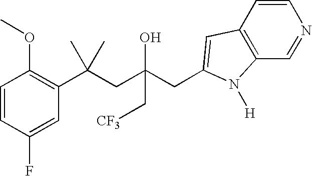 |
| 4-(5-Fluoro-2-methoxyphenyl)-4-methyl- 2-phenyl-1-(1H-pyrrolo[2,3-c]pyridin-2- yl)pentan-2-ol | 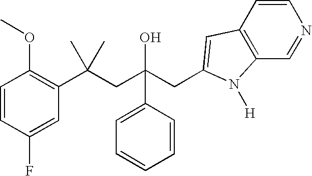 |
| 5-(5-Chloro-2,3-dihydrobenzofuran-7-yl)- 2,2,5-trimethyl-3-(1H-pyrrolo[2,3- c]pyridin-2-ylmethyl)hexan-3-ol | 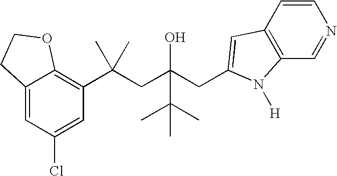 |
| 5-(5-Fluoro-2-methylphenyl)-2,2,5- trimethyl-3-thieno[2,3-c]pyridin-2- ylmethylhexan-3-ol | 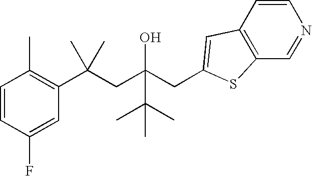 |
| 1,1-Difluoro-4-(5-fluoro-2- methoxyphenyl)-4-methyl-2-(1H- pyrrolo[3,2-c]pyridin-2-ylmethyl)pentan- 2-ol | 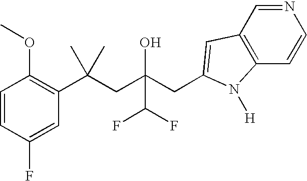 |
| 5-(5-Fluoro-2-methoxyphenyl)-2,5- dimethyl-3-(1H-pyrrolo[3,2-c]pyridin-2- ylmethyl)hexan-3-ol | 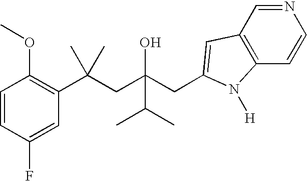 |
| 5-(5-Fluoro-2-methoxyphenyl)-2,2,5- trimethyl-3-(1H-pyrrolo[3,2-c]pyridin-2- ylmethyl)hexan-3-ol | 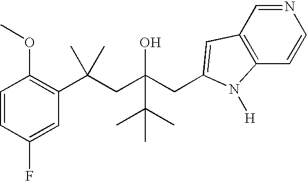 |
| 2-(1-Fluorocyclopropyl)-4-(5-fluoro-2- methoxyphenyl)-4-methyl-1-(1H- pyrrolo[2,3-c]pyridin-2-yl)pentan-2-ol | 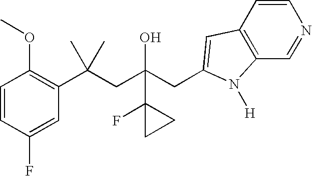 |
| 2-(1-Fluorocyclopropyl)-4-(4- fluorophenyl)-4-methyl-1-quinolin-4- ylpentan-2-ol | 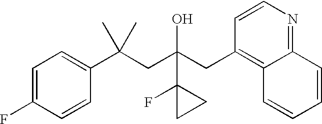 |

or a tautomer, prodrug, solvate, or salt thereof.

Preferred compounds of Formula (IB) include:
2-Cyclopropyl-4-(5-fluoro-2-methoxyphenyl)-4-methyl-1-(1H-pyrrolo[2,3-c]pyridin-2-yl)pentan-2-ol;
2-Cyclopropyl-4-(5-fluoro-2-methylphenyl)-4-methyl-1-(1H-pyrrolo [2,3-c]pyridin-2-yl)pentan-2-ol;
4-(5-Chloro-2,3-dihydrobenzofuran-7-yl)-2-cyclopropyl-4-methyl-1-(1H-pyrrolo [2,3-c]pyridin-2-yl)pentan-2-ol;
2-Cyclopropyl-4-(5-fluoro-2-methylphenyl)-4-methyl-1-(1H-pyrrolo[3,2-c]pyridin-2-yl)pentan-2-ol;
4-(5-Chloro-2,3-dihydrobenzofuran-7-yl)-2-cyclopropyl-4-methyl-1-(1H-pyrrolo[3,2-c]pyridin-2-yl)pentan-2-ol;
5-(5-Fluoro-2-methoxyphenyl)-2,5-dimethyl-3-(1H-pyrrolo[2,3-c]pyridin-2-ylmethyl)hexan-3-ol;
5-(5-Fluoro-2-methoxyphenyl)-2,2,5-trimethyl-3-(1H-pyrrolo[2,3-c]pyridin-2-ylmethyl)hexan-3-ol;
5-(5-Fluoro-2-methylphenyl)-2,5-dimethyl-3-(1H-pyrrolo[2,3-c]pyridin-2-ylmethyl)hexan-3-ol;
5-(5-Chloro-2,3-dihydrobenzofuran-7-yl)-2,5-dimethyl-3-(1H-pyrrolo[2,3-c]pyridin-2-ylmethyl)hexan-3-ol;
1,1-Difluoro-4-(5-fluoro-2-methoxyphenyl)-4-methyl-2-(1H-pyrrolo[3,2-c]pyridin-2-ylmethyl)pentan-2-ol;
5-(5-Fluoro-2-methoxyphenyl)-2,5-dimethyl-3-(1H-pyrrolo[3,2-c]pyridin-2-ylmethyl)hexan-3-ol; and
5-(5-Fluoro-2-methoxyphenyl)-2,2,5-trimethyl-3-(1H-pyrrolo[3,2-c]pyridin-2-ylmethyl)hexan-3-ol,
   or a tautomer, prodrug, solvate, or salt thereof.

In another aspect of the invention, the compounds according to the invention are formulated into pharmaceutical compositions comprising an effective amount, preferably a pharmaceutically effective amount, of a compound according to the invention or a tautomer, prodrug, solvate, or salt thereof, and a pharmaceutically acceptable excipient or carrier.

The invention also provides a method of inhibiting the aromatase enzyme in a patient, the method comprising administering to the patient an effective amount of a compound according to the invention or a tautomer, prodrug, solvate, or salt thereof.

The invention further provides a method of treating a disease-state or condition mediated by the aromatase enzyme in a patient in need of such treatment, the method comprising administering to the patient an effective amount of a pharmaceutically acceptable compound according to the invention or a tautomer, prodrug, solvate, or salt thereof.

In addition, the invention also provides a method of treating a disease-state or condition selected from estrogen receptor-positive breast cancer or benign conditions, such as cyclical breast pain, fibroadenomata, recurrent cystic disease, or endometriosis conditions such as gynaecomastia, or prostrate cancer in a patient in need of such treatment, the method comprising administering to the patient an effective amount of a pharmaceutically acceptable compound according to the invention or a tautomer, prodrug, solvate, or salt thereof.

The invention further provides methods of treating the disease-states or conditions mentioned above, in a patient in need of such treatment, the methods comprising sequentially or simultaneously administering to the patient: (a) an effective amount of a pharmaceutically acceptable compound according to the invention or a tautomer, prodrug, solvate, or salt thereof, and (b) a pharmaceutically acceptable aromatase inhibitor, tamoxifen, or other agents for hormonal therapy.

The invention further provides a method of assaying the aromatase enzyme function in a sample, comprising: (a) contacting the sample with a selected amount of a compound according to the invention or a tautomer, prodrug, solvate, or salt thereof, and (b) detecting the amount of the compound according to the invention or a tautomer, prodrug, solvate, or salt thereof necessary to inhibit the enzyme. In a preferred embodiment of the invention, the compound according to the invention or a tautomer, prodrug, solvate, or salt thereof is labeled with a detectable marker selected from: a radiolabel, fluorescent tag, a chemiluminescent tag, a chromophore, and a spin label.

The invention also provides a kit for the in vitro diagnostic determination of the aromatase enzyme function in a sample, comprising: (a) a diagnostically effective amount of a compound according to the invention or a tautomer, prodrug, solvate, or salt thereof; and (b) instructions for use of the diagnostic kit.

### DETAILED DESCRIPTION OF THE INVENTION

Definition of Terms and Conventions Used

Terms not specifically defined herein should be given the meanings that would be given to them by one of skill in the art in light of the disclosure and the context. As used in the specification and appended claims, however, unless specified to the contrary, the following terms have the meaning indicated and the following conventions are adhered to.

A. Chemical Nomenclature, Terms, and Conventions

In the groups, radicals, or moieties defined below, the number of carbon atoms is often specified preceding the group, for example, C₁-C₁₀ alkyl means an alkyl group or radical having 1 to 10 carbon atoms. The term “lower” applied to any carbon-containing group means a group containing from 1 to 8 carbon atoms, as appropriate to the group (i.e., a cyclic group must have at least 3 atoms to constitute a ring). In general, for groups comprising two or more subgroups, the last named group is the radical attachment point, for example, “alkylaryl” means a monovalent radical of the formula Alk-Ar-, while “arylalkyl” means a monovalent radical of the formula Ar-Alk- (where Alk is an alkyl group and Ar is an aryl group). Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups.

The terms “alkyl” or “alkyl group” mean a branched or straight-chain saturated aliphatic hydrocarbon monovalent radical. This term is exemplified by groups such as methyl, ethyl, n-propyl, 1-methylethyl (isopropyl), n-butyl, n-pentyl, 1,1-dimethylethyl (tert-butyl), and the like. It may be abbreviated “Alk”.

The terms “alkenyl” or “alkenyl group” mean a branched or straight-chain aliphatic hydrocarbon monovalent radical containing at least one carbon-carbon double bond. This term is exemplified by groups such as ethenyl, propenyl, n-butenyl, isobutenyl, 3-methylbut-2-enyl, n-pentenyl, heptenyl, octenyl, decenyl, and the like.

The terms “alkynyl” or “alkynyl group” mean a branched or straight-chain aliphatic hydrocarbon monovalent radical containing at least one carbon-carbon triple bond. This term is exemplified by groups such as ethynyl, propynyl, n-butynyl, 2-butynyl, 3-methylbutynyl, n-pentynyl, heptynyl, octynyl, decynyl, and the like.

The terms “alkylene” or “alkylene group” mean a branched or straight-chain saturated aliphatic hydrocarbon divalent radical having the specified number of carbon atoms. This term is exemplified by groups such as methylene, ethylene, propylene, n-butylene, and the like, and may alternatively and equivalently be denoted herein as -(alkyl)-.

The terms “alkenylene” or “alkenylene group” mean a branched or straight-chain aliphatic hydrocarbon divalent radical having the specified number of carbon atoms and at least one carbon-carbon double bond. This term is exemplified by groups such as ethenylene, propenylene, n-butenylene, and the like, and may alternatively and equivalently be denoted herein as -(alkylenyl)-.

The terms “alkynylene” or “alkynylene group” mean a branched or straight-chain aliphatic hydrocarbon divalent radical containing at least one carbon-carbon triple bond. This term is exemplified by groups such as ethynylene, propynylene, n-butynylene, 2-butynylene, 3methylbutynylene, n-pentynylene, heptynylene, octynylene, decynylene, and the like, and may alternatively and equivalently be denoted herein as -(alkynyl)-.

The terms “alkoxy” or “alkoxy group” mean a monovalent radical of the formula AlkO-, where Alk is an alkyl group. This term is exemplified by groups such as methoxy, ethoxy, propoxy, isopropoxy, butoxy, sec-butoxy, tert-butoxy, pentoxy, and the like.

The terms “aryloxy”, “aryloxy group”, mean a monovalent radical of the formula ArO—, where Ar is aryl. This term is exemplified by groups such as phenoxy, naphthoxy, and the like.

The terms “alkylcarbonyl”, “alkylcarbonyl group”, “alkanoyl”, or “alkanoyl group” mean a monovalent radical of the formula AlkC(O)—, where Alk is alkyl or hydrogen.

The terms “arylcarbonyl”, “arylcarbonyl group”, “aroyl” or “aroyl group” mean a monovalent radical of the formula ArC(O)—, where Ar is aryl.

The terms “acyl” or “acyl group” mean a monovalent radical of the formula RC(O)—, where R is a substituent selected from hydrogen or an organic substituent. Exemplary substituents include alkyl, aryl, arylalkyl, cycloalkyl, heterocyclyl, heteroaryl, heteroarylalkyl, and the like. As such, the terms comprise alkylcarbonyl groups and arylcarbonyl groups.

The terms “acylamino” or “acylamino group” mean a monovalent radical of the formula RC(O)N(R)—, where each R is a substituent selected from hydrogen or a substituent group.

The terms “alkoxycarbonyl” or “alkoxycarbonyl group” mean a monovalent radical of the formula AlkO-C(O)—, where Alk is alkyl. Exemplary alkoxycarbonyl groups include methoxycarbonyl, ethoxycarbonyl, tert-butyloxycarbonyl, and the like.

The terms “alkylaminocarbonyloxy” or “alkylaminocarbonyloxy group” mean a monovalent radical of the formula R₂NC(O)O—, where each R is independently hydrogen or lower alkyl.

The term “alkoxycarbonylamino” or “alkoxycarbonylamino group” mean a monovalent radical of the formula ROC(O)NH—, where R is lower alkyl.

The terms “alkylcarbonylamino” or “alkylcarbonylamino group” or “alkanoylamino” or “alkanoylamino groups” mean a monovalent radical of the formula AlkC(O)NH—, where Alk is alkyl. Exemplary alkylcarbonylamino groups include acetamido (CH₃C(O)NH—).

The terms “alkylaminocarbonyloxy” or “alkylaminocarbonyloxy group” mean a monovalent radical of the formula AlkNHC(O)O—, where Alk is alkyl.

The terms “amino” or “amino group” mean an —NH₂ group.

The terms “alkylamino” or “alkylamino group” mean a monovalent radical of the formula (Alk)NH—, where Alk is alkyl. Exemplary alkylamino groups include methylamino, ethylamino, propylamino, butylamino, tert-butylamino, and the like.

The terms “dialkylamino” or “dialkylamino group” mean a monovalent radical of the formula (Alk)(Alk)N—, where each Alk is independently alkyl. Exemplary dialkylamino groups include dimethylamino, methylethylamino, diethylamino, dipropylamino, ethylpropylamino, and the like.

The terms “substituted amino” or “substituted amino group” mean a monovalent radical of the formula —NR₂, where each R is independently a substituent selected from hydrogen or the specified substituents (but where both Rs cannot be hydrogen). Exemplary substituents include alkyl, alkanoyl, aryl, arylalkyl, cycloalkyl, heterocyclyl, heteroaryl, heteroarylalkyl, and the like.

The terms “alkoxycarbonylamino” or “alkoxycarbonylamino group” mean a monovalent radical of the formula AlkOC(O)NH—, where Alk is alkyl.

The terms “ureido” or “ureido group” mean a monovalent radical of the formula R₂NC(O)NH—, where each R is independently hydrogen or alkyl.

The terms “halogen” or “halogen group” mean a fluoro, chloro, bromo, or iodo group.

The term “halo” means one or more hydrogen atoms of the group are replaced by halogen groups.

The terms “haloalkyl” or “haloalkyl group” mean a branched or straight-chain saturated aliphatic hydrocarbon monovalent radical, wherein one or more hydrogen atoms thereof are each independently replaced with halogen atoms. This term is exemplified by groups such as chloromethyl, 1,2-dibromoethyl, 1,1,1-trifluoropropyl, 2-iodobutyl, 1-chloro-2-bromo-3-fluoropentyl, and the like.

The terms “sulfanyl”, “sulfanyl group”, “thioether”, or “thioether group” mean a divalent radical of the formula —S—.

The terms “alkylthio” or “alkylthio group” mean a monovalent radical of the formula AlkS-, where Alk is alkyl. Exemplary groups include methylthio, ethylthio, n-propylthio, isopropylthio, n-butylthio, and the like.

The terms “sulfonyl” or “sulfonyl group” mean a divalent radical of the formula —SO₂—.

The terms “sulfonylamino” or “sulfonylamino group” mean a divalent radical of the formula —SO₂NR—, where R is a hydrogen or a substituent group.

The terms “aminosulfonyl” or “aminosulfonyl group” mean a monovalent radical of the formula NR₂SO₂—, where R is each independently a hydrogen or a substituent group.

The terms “carbocycle” or “carbocyclic group” mean a stable aliphatic 3- to 15-membered monocyclic or polycyclic monovalent or divalent radical consisting solely of carbon and hydrogen atoms which may comprise one or more fused or bridged ring(s), preferably a 5- to 7-membered monocyclic or 7- to 10-membered bicyclic ring. Unless otherwise specified, the carbocycle may be attached at any carbon atom which results in a stable structure and, if substituted, may be substituted at any suitable carbon atom which results in a stable structure. The term comprises cycloalkyl (including spiro cycloalkyl), cycloalkylene, cycloalkenyl, cycloalkenylene, cycloalkynyl, and cycloalkynylene, and the like.

The terms “cycloalkyl” or “cycloalkyl group” mean a stable aliphatic saturated 3- to 15-membered monocyclic or polycyclic monovalent radical consisting solely of carbon and hydrogen atoms which may comprise one or more fused or bridged ring(s), preferably a 5- to 7-membered monocyclic or 7- to 10-membered bicyclic ring. Unless otherwise specified, the cycloalkyl ring may be attached at any carbon atom which results in a stable structure and, if substituted, may be substituted at any suitable carbon atom which results in a stable structure. Exemplary cycloalkyl groups include cyclopropyl, cyclobutyl, cyclopentyl, cyclohexyl, cycloheptyl, cyclooctyl, cyclononyl, cyclodecyl, norbornanyl, adamantyl, tetrahydronaphthyl (tetralin), 1-decalinyl, bicyclo[2.2.2]octanyl, 1-methylcyclopropyl, 2-methylcyclopentyl, 2-methylcyclooctyl, and the like.

The terms “cycloalkenyl” or “cycloalkenyl group” mean a stable aliphatic 3- to 15-membered monocyclic or polycyclic monovalent radical having at least one carbon-carbon double bond and consisting solely of carbon and hydrogen atoms which may comprise one or more fused or bridged ring(s), preferably a 5- to 7-membered monocyclic or 7- to 10-membered bicyclic ring. Unless otherwise specified, the cycloalkenyl ring may be attached at any carbon atom which results in a stable structure and, if substituted, may be substituted at any suitable carbon atom which results in a stable structure. Exemplary cycloalkenyl groups include cyclopentenyl, cyclohexenyl, cycloheptenyl, cyclooctenyl, cyclononenyl, cyclodecenyl, norbornenyl, 2-methylcyclopentenyl, 2-methylcyclooctenyl, and the like.

The terms “cycloalkynyl” or “cycloalkynyl group” mean a stable aliphatic 8- to 15-membered monocyclic or polycyclic monovalent radical having at least one carbon-carbon triple bond and consisting solely of carbon and hydrogen atoms which may comprise one or more fused or bridged ring(s), preferably a 8- to 10-membered monocyclic or 12- to 15-membered bicyclic ring. Unless otherwise specified, the cycloalkynyl ring may be attached at any carbon atom which results in a stable structure and, if substituted, may be substituted at any suitable carbon atom which results in a stable structure. Exemplary cycloalkynyl groups include, cyclooctynyl, cyclononynyl, cyclodecynyl, 2-methylcyclooctynyl, and the like.

The terms “cycloalkylene” or “cycloalkylene group” mean a stable saturated aliphatic 3- to 15-membered monocyclic or polycyclic divalent radical consisting solely of carbon and hydrogen atoms which may comprise one or more fused or bridged ring(s), preferably a 5- to 7-membered monocyclic or 7- to 10-membered bicyclic ring. Unless otherwise specified, the cycloalkyl ring may be attached at any carbon atom which results in a stable structure and, if substituted, may be substituted at any suitable carbon atom which results in a stable structure. Exemplary cycloalkylene groups include cyclopentylene, and the like.

The terms “cycloalkenylene” or “cycloalkenylene group” mean a stable aliphatic 5- to 15-membered monocyclic or polycyclic divalent radical having at least one carbon-carbon double bond and consisting solely of carbon and hydrogen atoms which may comprise one or more fused or bridged ring(s), preferably a 5- to 7-membered monocyclic or 7- to 10-membered bicyclic ring. Unless otherwise specified, the cycloalkenylene ring may be attached at any carbon atom which results in a stable structure and, if substituted, may be substituted at any suitable carbon atom which results in a stable structure. Exemplary cycloalkenylene groups include cyclopentenylene, cyclohexenylene, cycloheptenylene, cyclooctenylene, cyclononenylene, cyclodecenylene, norbornenylene, 2-methylcyclopentenylene, 2-methylcyclooctenylene, and the like.

The terms “cycloalkynylene” or “cycloalkynylene group” mean a stable aliphatic 8- to 15-membered monocyclic or polycyclic divalent radical having at least one carbon-carbon triple bond and consisting solely of carbon and hydrogen atoms which may comprise one or more fused or bridged ring(s), preferably a 8- to 10-membered monocyclic or 12- to 15-membered bicyclic ring. Unless otherwise specified, the cycloalkynylene ring may be attached at any carbon atom which results in a stable structure and, if substituted, may be substituted at any suitable carbon atom which results in a stable structure. Exemplary cycloalkynylene groups include cyclooctynylene, cyclononynylene, cyclodecynylene, 2-methylcyclooctynylene, and the like.

The terms “aryl” or “aryl group” mean an aromatic carbocyclic monovalent or divalent radical of from 6 to 14 carbon atoms having a single ring (e.g., phenyl or phenylene) or multiple condensed rings (e.g., naphthyl or anthranyl). Unless otherwise specified, the aryl ring may be attached at any suitable carbon atom which results in a stable structure and, if substituted, may be substituted at any suitable carbon atom which results in a stable structure. Exemplary aryl groups include phenyl, naphthyl, anthryl, phenanthryl, indanyl, indenyl, biphenyl, and the like. It may be abbreviated “Ar”.

The terms “heteroaryl” or “heteroaryl group” mean a stable aromatic 5- to 14-membered, monocyclic or polycyclic monovalent or divalent radical which may comprise one or more fused or bridged ring(s), preferably a 5- to 7-membered monocyclic or 7- to 10-membered bicyclic radical, having from one to four heteroatoms in the ring(s) independently selected from nitrogen, oxygen, and sulfur, wherein any sulfur heteroatoms may optionally be oxidized and any nitrogen heteroatom may optionally be oxidized or be quaternized. Unless otherwise specified, the heteroaryl ring may be attached at any suitable heteroatom or carbon atom which results in a stable structure and, if substituted, may be substituted at any suitable heteroatom or carbon atom which results in a stable structure. Exemplary and preferred heteroaryls include furanyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, triazinyl, indolizinyl, azaindolizinyl, indolyl, azaindolyl, diazaindolyl, dihydroindolyl, dihydroazaindoyl, isoindolyl, azaisoindolyl, benzofuranyl, furanopyridinyl, furanopyrimidinyl, furanopyrazinyl, furanopyridazinyl, dihydrobenzofuranyl, dihydrofuranopyridinyl, dihydrofuranopyrimidinyl, benzodioxolanyl, benzothienyl, thienopyridinyl, thienopyrimidinyl, thienopyrazinyl, thienopyridazinyl, dihydrobenzothienyl, dihydrothienopyridinyl, dihydrothienopyrimidinyl, indazolyl, azaindazolyl, diazaindazolyl, benzimidazolyl, imidazopyridinyl, benzthiazolyl, thiazolopyridinyl, thiazolopyrimidinyl, benzoxazolyl, oxazolopyridinyl, oxazolopyrimidinyl, benzisoxazolyl, purinyl, chromanyl, azachromanyl, quinolizinyl, quinolinyl, dihydroquinolinyl, tetrahydroquinolinyl, isoquinolinyl, dihydroisoquinolinyl, tetrahydroisoquinolinyl, cinnolinyl, azacinnolinyl, phthalazinyl, azaphthalazinyl, quinazolinyl, azaquinazolinyl, quinoxalinyl, azaquinoxalinyl, naphthyridinyl, dihydronaphthyridinyl, tetrahydronaphthyridinyl, pteridinyl, carbazolyl, acridinyl, phenazinyl, phenothiazinyl, and phenoxazinyl, and the like.

The terms “heterocycle”, “heterocycle group”, “heterocyclyl”, or “heterocyclyl group” mean a stable non-aromatic 5- to 14-membered monocyclic or polycyclic, monovalent or divalent, ring which may comprise one or more fused or bridged ring(s), preferably a 5- to 7-membered monocyclic or 7- to 10-membered bicyclic ring, having from one to three heteroatoms in the ring(s) independently selected from nitrogen, oxygen, and sulfur, wherein any sulfur heteroatoms may optionally be oxidized and any nitrogen heteroatom may optionally be oxidized or be quaternized. Unless otherwise specified, the heterocyclyl ring may be attached at any suitable heteroatom or carbon atom which results in a stable structure and, if substituted, may be substituted at any suitable heteroatom or carbon atom which results in a stable structure. Exemplary and preferred heterocycles include pyrrolinyl, pyrrolidinyl, pyrazolinyl, pyrazolidinyl, piperidinyl, morpholinyl, thiomorpholinyl, piperazinyl, tetrahydropyranyl, tetrahydrothiopyranyl, tetrahydrofuranyl, hexahydropyrimidinyl, hexahydropyridazinyl, and the like.

The term “compounds of Formula (I)” and equivalent expressions are mean to embrace either or both of compounds of Formula (IA) and compounds of Formula (IB) as the context permits.

The term “compounds of the invention” and equivalent expressions are meant to embrace compounds of Formula (I) as herein described, including the tautomers, the prodrugs, the salts, particularly the pharmaceutically acceptable salts, and the solvates and hydrates thereof, where the context so permits. In general and preferably, the compounds of the invention and the formulas designating the compounds of the invention are understood to only include the stable compounds thereof and exclude unstable compounds, even if an unstable compound might be considered to be literally embraced by the compound formula. Similarly, reference to intermediates, whether or not they themselves are claimed, is meant to embrace their salts and solvates, where the context so permits. For the sake of clarity, particular instances when the context so permits are sometimes indicated in the text, but these instances are purely illustrative and it is not intended to exclude other instances when the context so permits.

The terms “optional” or “optionally” mean that the subsequently described event or circumstances may or may not occur, and that the description includes instances where the event or circumstance occurs and instances in which it does not. For example, “optionally substituted aryl” means that the aryl radical may or may not be substituted and that the description includes both substituted aryl radicals and aryl radicals having no substitution.

The terms “stable compound” or “stable structure” mean a compound that is sufficiently robust to survive isolation to a useful degree of purity from a reaction mixture, and formulation into an efficacious therapeutic or diagnostic agent. For example, a compound which would have a “dangling valency” or is a carbanion is not a compound contemplated by the invention.

The term “substituted” means that any one or more hydrogens on an atom of a group or moiety, whether specifically designated or not, is replaced with a selection from the indicated group of substituents, provided that the atom's normal valency is not exceeded and that the substitution results in a stable compound. If a bond to a substituent is shown to cross the bond connecting two atoms in a ring, then such substituent may be bonded to any atom on the ring. When a substituent is listed without indicating the atom via which such substituent is bonded to the rest of the compound, then such substituent may be bonded via any atom in such substituent. For example, when the substituent is piperazinyl, piperidinyl, or tetrazolyl, unless specified otherwise, such piperazinyl, piperidinyl, or tetrazolyl group may be bonded to the rest of the compound of the invention via any atom in such piperazinyl, piperidinyl, or tetrazolyl group. Generally, when any substituent or group occurs more than one time in any constituent or compound, its definition on each occurrence is independent of its definition at every other occurrence. Thus, for example, if a group is shown to be substituted with 0 to 2 R⁵, then such group is optionally substituted with up to two R⁵ groups and R⁵ at each occurrence is selected independently from the defined list of possible R⁵. Such combinations of substituents and/or variables, however, are permissible only if such combinations result in stable compounds.

In a specific embodiment, the term “about” or “approximately” means within 20%, preferably within 10%, and more preferably within 5% of a given value or range.

The yield of each of the reactions described herein is expressed as a percentage of the theoretical yield.

B. Salt, Prodrug, Derivative, and Solvate Terms and Conventions

The terms “prodrug” or “prodrug derivative” mean a covalently-bonded derivative or carrier of the parent compound or active drug substance which undergoes at least some biotransformation prior to exhibiting its pharmacological effect(s). In general, such prodrugs have metabolically cleavable groups and are rapidly transformed in vivo to yield the parent compound, for example, by hydrolysis in blood, and generally include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309-396; Burger's Medicinal Chemistry and Drug Discovery, 5th Ed., M. Wolff (ed.), John Wiley & Sons, 1995, particularly Vol. 1 and pp. 172-178 and pp. 949-982; Pro-Drugs as Novel Delivery Systems, T. Higuchi and V. Stella (eds.), Am. Chem. Soc., 1975; Bioreversible Carriers in Drug Design, E. B. Roche (ed.), Elsevier, 1987, each of which is incorporated herein by reference in their entireties.

The term “pharmaceutically acceptable prodrug” as used herein means a prodrug of a compound of the invention which is, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible.

The term “salt” means an ionic form of the parent compound or the product of the reaction between the parent compound with a suitable acid or base to make the acid salt or base salt of the parent compound. Salts of the compounds of the present invention can be synthesized from the parent compounds which contain a basic or acidic moiety by conventional chemical methods. Generally, the salts are prepared by reacting the free base or acid parent compound with stoichiometric amounts or with an excess of the desired salt-forming inorganic or organic acid or base in a suitable solvent or various combinations of solvents.

The term “pharmaceutically acceptable salt” means a salt of a compound of the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. As the compounds of the present invention are useful in both free base and salt form, in practice, the use of the salt form amounts to use of the base form. Lists of suitable salts are found in, e.g., S. M. Birge et al., J. Pharm. Sci., 1977, 66, pp. 1-19, which is hereby incorporated by reference in its entirety.

The term “pharmaceutically-acceptable acid addition salt” means those salts which retain the biological effectiveness and properties of the free bases and which are not biologically or otherwise undesirable, formed with inorganic acids such as hydrochloric acid, hydrobromic acid, hydroiodic acid, sulfuric acid, sulfamic acid, nitric acid, phosphoric acid, and the like, and organic acids such as acetic acid, trichloroacetic acid, trifluoroacetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, heptanoic acid, hexanoic acid, formic acid, fumaric acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, undecanoic acid, and the like.

The term “pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-acceptable organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like. Particularly preferred organic nontoxic bases are isopropylamine, diethylamine, ethanolamine, trimethylamine, dicyclohexylamine, choline, and caffeine.

The term “solvate” means a physical association of a compound with one or more solvent molecules or a complex of variable stoichiometry formed by a solute (for example, a compound of Formula (I)) and a solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, the solvents selected do not interfere with the biological activity of the solute. Solvates encompasses both solution-phase and isolatable solvates. Representative solvates include hydrates, ethanolates, methanolates, and the like.

The term “hydrate” means a solvate wherein the solvent molecule(s) is/are H₂O.

The compounds of the present invention as discussed below include the free base or acid thereof, their salts, solvates, and prodrugs and may include oxidized sulfur atoms or quaternized nitrogen atoms in their structure, although not explicitly stated or shown, particularly the pharmaceutically acceptable forms thereof. Such forms, particularly the pharmaceutically acceptable forms, are intended to be embraced by the appended claims.

C. Isomer Terms and Conventions

The term “isomers” means compounds having the same number and kind of atoms, and hence the same molecular weight, but differing with respect to the arrangement or configuration of the atoms in space. The term includes stereoisomers and geometric isomers.

The terms “stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the invention which may give rise to stereoisomerism, the invention contemplates stereoisomers and mixtures thereof. The compounds of the invention and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

The term “enantiomers” means a pair of stereoisomers that are non-superimposable mirror images of each other.

The terms “diastereoisomers” or “diastereomers” mean optical isomers which are not mirror images of each other.

The terms “racemic mixture” or “racemate” mean a mixture containing equal parts of individual enantiomers.

The term “non-racemic mixture” means a mixture containing unequal parts of individual enantiomers.

The term “geometrical isomer” means a stable isomer which results from restricted freedom of rotation about double bonds (e.g., cis-2-butene and trans-2-butene) or in a cyclic structure (e.g., cis-1,3-dichlorocyclobutane and trans-1,3-dichlorocyclobutane). Because carbon-carbon double (olefinic) bonds, C═N double bonds, cyclic structures, and the like may be present in the compounds of the invention, the invention contemplates each of the various stable geometric isomers and mixtures thereof resulting from the arrangement of substituents around these double bonds and in these cyclic structures. The substituents and the isomers are designated using the cis/trans convention or using the E or Z system, wherein the term “E” means higher order substituents on opposite sides of the double bond, and the term “Z” means higher order substituents on the same side of the double bond. A thorough discussion of E and Z isomerism is provided in J. March, Advanced Organic Chemistry: Reactions, Mechanisms, and Structure, 4th ed., John Wiley & Sons, 1992, which is hereby incorporated by reference in its entirety. Several of the following examples represent single E isomers, single Z isomers, and mixtures of E/Z isomers. Determination of the E and Z isomers can be done by analytical methods such as x-ray crystallography, ¹H NMR, and ¹³C NMR.

Some of the compounds of the invention can exist in more than one tautomeric form. As mentioned above, the compounds of the invention include all such tautomers.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the invention from this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

Thus, if one enantiomer is pharmacologically more active, less toxic, or has a preferred disposition in the body than the other enantiomer, it would be therapeutically more beneficial to administer that enantiomer preferentially. In this way, the patient undergoing treatment would be exposed to a lower total dose of the drug and to a lower dose of an enantiomer that is possibly toxic or an inhibitor of the other enantiomer.

Preparation of pure enantiomers or mixtures of desired enantiomeric excess (ee) or enantiomeric purity are accomplished by one or more of the many methods of (a) separation or resolution of enantiomers, or (b) enantioselective synthesis known to those of skill in the art, or a combination thereof. These resolution methods generally rely on chiral recognition and include, for example, chromatography using chiral stationary phases, enantioselective host-guest complexation, resolution or synthesis using chiral auxiliaries, enantioselective synthesis, enzymatic and nonenzymatic kinetic resolution, or spontaneous enantioselective crystallization. Such methods are disclosed generally in Chiral Separation Techniques: A Practical Approach (2nd Ed.), G. Subramanian (ed.), Wiley-VCH, 2000; T. E. Beesley and R. P. W. Scott, Chiral Chromatography, John Wiley & Sons, 1999; and Satinder Ahuja, Chiral Separations by Chromatography, Am. Chem. Soc., 2000. Furthermore, there are equally well-known methods for the quantitation of enantiomeric excess or purity, for example, GC, HPLC, CE, or NMR, and assignment of absolute configuration and conformation, for example, CD ORD, X-ray crystallography, or NMR.

In general, all tautomeric forms and isomeric forms and mixtures, whether individual geometric isomers or stereoisomers or racemic or non-racemic mixtures, of a chemical structure or compound is intended, unless the specific stereochemistry or isomeric form is specifically indicated in the compound name or structure.

D. Pharmaceutical Administration and Diagnostic and Treatment Terms and Conventions

The term “patient” includes both human and non-human mammals.

The term “effective amount” means an amount of a compound according to the invention which, in the context of which it is administered or used, is sufficient to achieve the desired effect or result. Depending on the context, the term effective amount may include or be synonymous with a pharmaceutically effective amount or a diagnostically effective amount.

The terms “pharmaceutically effective amount” or “therapeutically effective amount” means an amount of a compound according to the invention which, when administered to a patient in need thereof, is sufficient to effect treatment for disease-states, conditions, or disorders for which the compounds have utility. Such an amount would be sufficient to elicit the biological or medical response of a tissue, system, or patient that is sought by a researcher or clinician. The amount of a compound of according to the invention which constitutes a therapeutically effective amount will vary depending on such factors as the compound and its biological activity, the composition used for administration, the time of administration, the route of administration, the rate of excretion of the compound, the duration of treatment, the type of disease-state or disorder being treated and its severity, drugs used in combination with or coincidentally with the compounds of the invention, and the age, body weight, general health, sex, and diet of the patient. Such a therapeutically effective amount can be determined routinely by one of ordinary skill in the art having regard to their own knowledge, the prior art, and this disclosure.

The term “diagnostically effective amount” means an amount of a compound according to the invention which, when used in a diagnostic method, apparatus, or assay, is sufficient to achieve the desired diagnostic effect or the desired biological activity necessary for the diagnostic method, apparatus, or assay. Such an amount would be sufficient to elicit the biological or medical response in a diagnostic method, apparatus, or assay, which may include a biological or medical response in a patient or in a in vitro or in vivo tissue or system, that is sought by a researcher or clinician. The amount of a compound according to the invention which constitutes a diagnostically effective amount will vary depending on such factors as the compound and its biological activity, the diagnostic method, apparatus, or assay used, the composition used for administration, the time of administration, the route of administration, the rate of excretion of the compound, the duration of administration, drugs and other compounds used in combination with or coincidentally with the compounds of the invention, and, if a patient is the subject of the diagnostic administration, the age, body weight, general health, sex, and diet of the patient. Such a diagnostically effective amount can be determined routinely by one of ordinary skill in the art having regard to their own knowledge, the prior art, and this disclosure.

It should be noted that he present invention includes compounds and compositions in which a compound of the invention is either combined with, or covalently bound to, a cytotoxic agent bound to a targeting agent, such as a monoclonal antibody (e.g., a murine or humanized monoclonal antibody). It will be appreciated that the latter combination may allow the introduction of cytotoxic agents into cancer cells with greater specificity. Thus, the active form of the cytotoxic agent (i.e., the free form) will be present only in cells targeted by the antibody. Of course, the compounds of the invention may also be combined with monoclonal antibodies that have therapeutic activity against cancer.

The term “estrogen receptor-mediated disorder” means any biological or medical disorder in which estrogen receptor activity is implicated or in which the inhibition of estrogen receptor potentiates or retards signaling through a pathway that is characteristically defective in the disease to be treated. The condition or disorder may either be caused or characterized by abnormal estrogen receptor activity. Representative estrogen receptor-mediated disorders include, for example, osteoporosis, atherosclerosis, estrogen-mediated cancers (e.g., breast and endometrial cancer), Turner's syndrome, benign prostate hyperplasia (i.e., prostate enlargement), prostate cancer, elevated cholesterol, restenosis, endometriosis, uterine fibroid disease, skin and/or vagina atrophy, and Alzheimer's disease. Successful treatment of a subject in accordance with the invention may result in the prevention, inducement of a reduction in, or alleviation of symptoms in a subject afflicted with an estrogen receptor-mediated medical or biological disorder. Thus, for example, treatment can result in a reduction in breast or endometrial tumors and/or various clinical markers associated with such cancers. Likewise, treatment of Alzheimer's disease can result in a reduction in rate of disease progression, detected, for example, by measuring a reduction in the rate of increase of dementia.

The terms “treating” or “treatment” mean the treatment of an estrogen receptor-mediated disorder or disease-state in a patient, and include:
(i) preventing or alleviating the estrogen receptor-mediated disorder or disease-state from occurring in a patient, in particular, when such patient is genetically or otherwise predisposed to the estrogen receptor-mediated disorder or disease-state but has not yet been diagnosed as having it;
(ii) inhibiting or ameliorating the estrogen receptor-mediated disorder or disease-state in a patient, i.e., arresting or slowing its development; or
(iii) relieving the estrogen receptor-mediated disorder or disease-state in a patient, i.e., causing regression or cure of the estrogen receptor-mediated disorder or disease-state.
   E. Combination Therapy Treatment Terms and Conventions

While the compounds of the invention can be administered as the sole active pharmaceutical agent, they can also be used in combination with one or more additional therapeutic agents as described herein, and/or in combination with other therapeutic agents used in the treatment and/or prevention of estrogen receptor-mediated disorders. Alternatively, the compounds of the present invention can be administered sequentially with one or more additional therapeutic agents to provide sustained therapeutic and prophylactic effects.

The term “additional therapeutic agent” means an agent that has been used in the treatment of estrogen receptor-mediated disorders or any anticancer or antineoplastic agent, especially those mentioned specifically herein.

The term “cotherapy” or “combination therapy”, in defining use of a compound of the invention and additional therapeutic agent, is intended to embrace administration of each agent in a sequential manner in a regimen that will provide beneficial effects of the drug combination, and is intended as well to embrace coadministration of these agents in a substantially simultaneous manner, such as in a single capsule having a fixed ratio of these active agents or in multiple, separate pharmaceutical forms for each agent.

In accordance with yet other embodiments, the present invention provides methods for treating or preventing an estrogen receptor-mediated disorder in a patient in which an amount of an estrogen receptor-modulating compound of the invention that is effective to modulate estrogen receptor activity in the subject. Other embodiments provided methods for treating a cell or a estrogen receptor-mediated disorder in a patient, comprising administering to the cell or to the patient an amount of a compound or composition of the invention effective to modulate estrogen receptor activity in the cell or subject. Preferably, the subject will be a human or non-human animal subject. Modulation of estrogen receptor activity detectable suppression or up-regulation of estrogen receptor activity either as compared to a control or as compared to expected estrogen receptor activity.

In combining the compound of the invention and the additional therapeutic agent, the administration time of the compound of the present invention and the additional therapeutic agent is not restricted, and the compound of the present invention and the additional therapeutic agent can be administered to an administration subject simultaneously, or may be administered at different times. The dosage of the additional therapeutic agent may be determined according to the administration amount clinically used, and can be appropriately selected depending on an administration subject, administration route, disease, combination and the like.

The administration mode of the compounds of the invention and the additional therapeutic agent of the present invention is not particularly restricted, and it is sufficient that the compound of the invention and the additional therapeutic agent are combined in administration. Examples of such administration mode include the following methods: (1) the compound of the invention and the additional therapeutic agent are simultaneously produced to give a single preparation which is administered; (2) the compound of the invention and the additional therapeutic agent are separately produced to give two kinds of preparations which are administered simultaneously by the same administration route; (3) the compound of the present invention and the additional therapeutic agent are separately produced to give two kinds of preparations which are administered by the same administration route at different times; (4) the compound of the invention and the additional therapeutic agent are separately produced to give two kinds of preparations which are administered simultaneously by different administration routes; or (5) the compound of the invention and the additional therapeutic agent are separately produced to give two kinds of preparations which are administered by different administration routes at different times (for example, the compound of the invention and the additional therapeutic agent are administered in this order, or in the reverse order).

In addition, the compounds of the invention can be used, either singly or in combination as described above, in combination with other modalities for preventing or treating. Such other treatment modalities include, without limitation, surgery, radiation. These can be performed.

Several classes of anticancer agents have been used to treat estrogen receptor-mediated diseases or disorders, particularly breast cancer, including selective estrogen-receptor modulators, aromatase inhibitors, biologic response modifiers, and other hormone therapy agents, and chemotherapy agents such as anthracyclines, taxanes, alkylating agents, antimetabolites antagonists, anticancer antibiotics, and plant-derived anticancer agents. It is known to those of skill in the art that such additional therapeutic agents, as well as surgery, radiation therapy, hormone supplementation, diet regulation, and adjunct therapy may be used in various combinations, either sequentially (e.g., treatment with a compound of the invention following surgery or radiation) or simultaneously (e.g., in addition to a diet regimen), in order to achieve an optimal therapeutic result in the patient.

The additional therapeutic agents may generally be employed in therapeutic amounts as indicated in the PHYSICIANS' DESK REFERENCE (PDR) 53rd Edition (1999), which is incorporated herein by reference, or such therapeutically useful amounts as would be known to one of ordinary skill in the art. The compounds of the invention and the additional therapeutic agents can be administered at the recommended maximum clinical dosage or at lower doses. Dosage levels of the active compounds in the compositions of the invention may be varied to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions that are given at the same time or different times, or the therapeutic agents can be given as a single composition.

The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The prophylactically or therapeutically effective amount for a given situation can be readily determined by routine experimentation and is within the skill and judgment of the ordinary clinician. For exemplary purposes of the present invention, a prophylactically or therapeutically effective dose will generally be from about 0.1 mg/kg/day to about 100 mg/kg/day, preferably from about 1 mg/kg/day to about 20 mg/kg/day, and most preferably from about 2 mg/kg/day to about 10 mg/kg/day of a estrogen receptor-modulating compound of the present invention, which may be administered in one or multiple doses.

There are large numbers of anticancer or antineoplastic agents available in commercial use, in clinical evaluation and in preclinical development, which would be selected for treatment of neoplasia by combination drug chemotherapy. These chemotherapeutic agents may be roughly categorized by their mechanism of action into, for example, following groups: anti-metabolites/anti-cancer agents, such as pyrimidine analogs (e.g., 5-fluorouracil, floxuridine, capecitabine, gemcitabine, and cytarabine) and purine analogs, folate antagonists, and related inhibitors (mercaptopurine, thioguanine, pentostatin, and 2-chlorodeoxyadenosine (cladribine)); antiproliferative/antimitotic agents including natural products such as vinca alkaloids (e.g., vinblastine, vincristine, and vinorelbine), microtubule disruptors such as taxanes (e.g., paclitaxel or docetaxel), vincristine, vinblastine, nocodazole, the epothilones, and navelbine, epidipodophyllotoxins (teniposide), DNA damaging agents (actinomycin, amsacrine, anthracyclines, bleomycin, busulfan, camptothecin, carboplatin, chlorambucil, cisplatin, cyclophosphamide, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, teniposide, triethylenethiophosphoramide, and etoposide); antibiotics such as dactinomycin (actinomycin D), daunorubicin, doxorubicin, idarubicin, anthracyclines, mitoxantrone, bleomycins, plicamycin (mithramycin), and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine and streptozocin), triazenes-darbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase, and urokinase), aspirin, COX-2 inhibitors, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); antiangiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; antisense oligonucleotides; antibodies (trastuzumab); cell cycle inhibitors and differentiation inducers (tretinoin); mTOR inhibitors, topoisomerase inhibitors (doxorubicin, amsacrine, camptothecin, daunorubicin, dactinomycin, eniposide, epirubicin, etoposide, idarubicin, mitoxantrone, topotecan, and irinotecan), corticosteroids (cortisone, dexamethasone, hydrocortisone, methylpednisolone, prednisone, and prednisolone); growth factor signal transduction kinase inhibitors; mitochondrial dysfunction inducers and caspase activators; chromatin disruptors. Although such anticancer agents may fall into several categories, several exemplary “families” of these agents are grouped below, one or more of which may be used in combination with the compounds of the invention.

A first family of anticancer agents which may be used in combination with compounds of the invention consists of aromatase inhibitors, selective estrogen-receptor modulators (SERMs), and other hormonal therapeutic agents. Aromatase inhibitors bind to aromatase and the many breast cancer cells that are estrogen-dependent are prevented from growing and dividing. There are several aromatase inhibitors that are used to help treat advanced breast cancer including: ARIMIDEX® (anastrozole), FEMARA® (letrozole), and AROMASIN® (exemestane). SERMs generally have a chemical resemblance to the hormone estrogen and are able to bind to estrogen receptors in breast cancer cells. By binding to these receptors, they block estrogen from breast cancer cells, thereby starving the cancer cells. Tamoxifen is currently the most commonly prescribed SERM and is approved by the FDA to treat both early and advanced stages of breast cancer and for use in postmenopausal women at high risk of breast cancer. FARESTON® (toremifene) is also used to treat advanced breast cancer. Another SERM, EVISTA® (raloxifene) is presently used to treat osteoporosis, a degenerative bone disease, however, raloxifene is currently being investigated to determine its effectiveness for treating breast cancer. In addition, other hormonal therapeutic agents are used to treat breast cancers that are dependent on estrogen for survival. For example, ZOLADEX® (goserelin acetate) is a synthetic form of lutenizing hormone-releasing hormone (LH-RH). Goserelin acetate blocks the release of estrogen in breast cancer patients (and testosterone in prostate cancer patients), preventing breast and prostate cancer cells from growing. Another hormone therapy, FASLODEX® (fulvestrant), appears to be effective for women who have become resistant to tamoxifen, according to recent studies. Instead of binding to estrogen receptors in breast cancer cells like tamoxifen, fulvestrant destroys estrogen receptors in cancer cells. Other hormonal therapeutic agents include, but are not limited to, fosfestrol, diethylstilbestrol (DES), estrogen, DEPO-PROVERA® (medroxyprogesterone acetate), PROVERA® (progestin/medroxyprogesterone acetate), PLENAXIS™ (abarelix), CYTADREN® (aminoglutethimide), AFEMA™ (fadrozole), Ligand Pharma LGD 122941, methyltestosterone, testosterone, testrolactone, abiraterone, allylestrenol, azoxifene, betamethasone, bicartamide, bromocriptine, chlorotrianisene, medroxyprogesterone, chlormadinone, clomiphene, cyproterone, danazol, deslorelin, droloxifene, enclomiphene, epristeride, epitiostanol, ethamoxytriphetol, ethinylestradiol, finasteride, flutamide, formestane, gestrinone, 4-hydroxytamoxifen, mepartricin, ormeloxifene, leuprorelin, levormeloxifene, mepitiostane, meterelin, nafarelin, nilutamide, dexamethasone, panomifene, prednisolone, triptorelin, turosteride, triamcinolone, liarozole, 4-hydroxytamoxifen, idoxifene, nafoxidene, delmadinone acetate, bisphosphonate, megestrol acetate, 4-hydroxyandrostenedione, plomestane, retrozole, rogletimide, vorozole, and zuclomiphene, or a tautomer, prodrug, solvate, or salt thereof.

A second family of anticancer or antineoplastic agents which may be used in combination with compounds of the invention consists of biologic response modifier agents. Biologic response modifiers bind with certain proteins on breast cancer cells, preventing their growth. HERCEPTIN® (trastuzumab) is a monoclonal antibody that attaches itself to HER2 (also written HER2/neu), a protein found on breast cancer cells. Approximately 30% of breast cancer patients have extra copies of the HER2 protein, which can signal more aggressive cancers. HERCEPTIN® binds to HER2 receptors on breast cancer cells, preventing them from growing and dividing. HERCEPTIN® is only indicated for breast cancer patients who overexpress the HER2 protein and patients should be tested for HER2 expression to determine whether HERCEPTIN® is a viable treatment option. Suitable biologic response modifier anticancer agents are, but not limited to, alemtuzumab, AVASTIN® (bevacizumab), ERBITUX® (cetuximab), daclizumab, dacliximab, edrecolomab, gemtuzumab, ibritumomab, lintuzumab, mitumomab, rituximab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-¹³¹I MAb (Techniclone), polymorphic epithelial mucin-⁹⁰Y MAb (Antisoma), and tositumomab-¹³¹I, or a tautomer, prodrug, solvate, or salt thereof.

A third family of anticancer or antineoplastic agents which may be used in combination with compounds of the invention consists of antimetabolites/DNA base analogs/thymidilate synthase inhibitor agents. Suitable antimetabolite anticancer agents are, but not limited to, 5-FU-fibrinogen, acanthifolic acid, acyclovir, adenine, adozelesin, aminothiadiazole, amethopterin, aminopterine, 2-aminopurine, ancitabine hydrochloride, aphidicolin, β-1-D-arabinoside, 8-azaguanine, azaserine, 6-azauracil, 2′-azido-2′-deoxynucleosides, brequinar sodium, 5-bromodeoxycytidine, butocine, capecitabine, Ciba-Geigy CGP-30694, cladribine, cyclopentyl cytosine, cytarabine, cytarabine ocfosfate, cytarabine phosphate stearate, cytarabine conjugates, cytosine, Lilly DATHF, Merrell Dow DDFC, decitabine, dezaguanine, diazooxynorleucine, dideoxycytidine, dideoxyguanosine, dideoxynucleosides, didox, Yoshitomi DMDC, doxifluridine, edatrexate, Wellcome EHNA, enocitabine, Merck EX-015, fazarabine, floxuridine, fludarabine, fludarabine phosphate, flurocitabine, 5fluorodeoxycytidine, 5-fluorodeoxyuridine, 5-fluorouracil (and tegafur, UFT, doxifluridine, carmofur, gallocitabine, and emitefur), folinate calcium, N-(2′-furanidyl)-5-fluorouracil, Daiichi Seiyaku FO-152, gemcitabine, hydroxycarbamide, hydroxyurea, idoxuridine, isopropyl pyrrolizine, lovastatin, Lilly LY-188011, Lilly LY-264618, methobenzaprim, leucovorin, levofolinate, loxoribine, mercaptopurine, 6-mercaptopurine riboside, methotrexate, methotrexate thioinosine, mitoguazone, Wellcome MZPES, nelarabine, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thiazophrine, thioguanine, tiazofurin, Erbamont TIF, raltitrexed, triciribine, trimetrexate, tyrosine protein kinase inhibitors, Taiho UFT, and uricytin, or a tautomer, prodrug, solvate, or salt thereof.

A fourth family of anticancer or antineoplastic agents which may be used in combination with compounds of the present invention consists of alkylating agents. Suitable alkylating agents and similar agents are, but are not limited to, Shionogi 254-S, adozelesin, aldo-phosphamide analogues, alkyl sulfonates, altretamine, ambamustine, anaxirone, atrimustine, azetepa, aziridines, Boehringer Mannheim BBR-2207, benzodepa, benzodizepa, bestrabucil, bizelesin, budotitane, busulfan, Wakunaga CA-102, carboplatin, carboquone, carmustine, Chinoin-139, Chinoin-153, chlorambucil, chlorambutyl, chlornaphazine, chlorozotocin, cisplatin, cyclophosphamide, American Cyanamid CL-286558, Sanofi CY-233, cyplatate, cystemustine, Degussa D-19-384, Sumimoto DACHP(Myr)₂, dacarbazine, dexormaplatin, dibrospidium hydrochloride, dimethyl sulfate, diphenylspiromustine, diplatinum cytostatic, Chugai DWA-2114R, ITI E09, ecomustine, elmustine, enloplatin, erythromycin, estramustine, estramustine phosphate sodium, ethylenimines, etoglucid, Erbamont FCE-24517, fotemustine, Unimed G-6-M, Chinoin GYKI-17230, hepsulfam, heptaplatin, ifosfamide, improsulfan, iproplatin, lomustine, mafosfamide, mannomustine, mechlorethamine, mechlorethamine oxide hydrochloride, melphalan, methylinelamines, N-methyl-N′-nitro-N-nitrosoguanidine (MNNG), N-methylscopolamine (NMS), meturedepa, miboplatin, mitobronitol, mitolactol, mitomycin C, nedaplatin, nimustine, nitrogen mustard, nitrogen mustard-N-oxide hydrochloride, nitroso ureas, Nippon Kayaku NK-121, novembichine, NCI NSC-264395, NCI NSC-342215, oxaliplatin, Upjohn PCNU, pentamustine, pipobroman, piposulfan, phenesterine, prednimustine, procarbazine, Proter PTT-119, pumitepa, ranimustine, ribomustin, satraplatin, semustine, SmithKline SK&F-101772, Yakult Honsha SN-22, spiromustine, spiroplatin, streptozocin, sulfur mustard, tallimustine, Tanabe Seiyaku TA-077, tauromustine, temozolomide, teroxirone, tetraplatin, thiotepa, treosulfan, triethylenemelamine, triethylenephosphoramide, triethylenethiophosphoramide, trimelamol, trimethylolinelamine trophosphamide, uracil mustard, uredepa, zeniplatin, and zinostatin stimalamer, or a tautomer, prodrug, solvate, or salt thereof.

A fifth family of anticancer or antineoplastic agents which may be used in combination with compounds of the present invention consists of antibiotic-type anticancer or antineoplastic agents. Suitable antibiotic-type anticancer agents are, but not limited to, Taiho 4181-A, aclarubicin, actinomycin-C, actinomycin-D, actinoplanone, acivicin, Erbamont ADR-456, aeroplysinin derivative, ambomycin, amrubicin, Ajinomoto AN-201-II, Ajinomoto AN-3, Nippon Soda anisomycins, ankinomycin, anthracycline, anthramycin, axinastatin 1, axinastatin 2, axinastatin 3, azinomycin-A, azotomycin, betaclamycin-B, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin, bryostatin-1, Taiho C-1027, cactinomycin, calichemycin, carubicin, carzinophilin, chromomycin-A3, chromoximycin, cirolemycin, collismycin-A, collismycin-B, combretastatin A4, coumermycin, cypemycin, cytostatin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, dioxamycin, distamycin, ditrisarubicin B, Shionogi DOB-41, doxorubicin, doxorubicin hydrochloride, doxorubicin-fibrinogen, duazomycin, elsamicin-A, epirubicin, epirubicin hydrochloride, erbstatin, esorubicin, esperamicin-A1, esperamicin-Alb, Erbamont FCE-21954, Fujisawa FK-973, fostriecin, Fujisawa FR-900482, glidobactin, gregatin-A, grincamycin, herbimycin, idarubicin, illudins, iododoxorubicin, kanamycin, kasugamycin, kazusamycin, kesarirhodins, kirromycin, Kyowa Hakko KM-5539, Kirin Brewery KRN-8602, Kyowa Hakko KT-5432, Kyowa Hakko KT-5594, Kyowa Hakko KT-6149, leinamycin, leptolstatin, American Cyanamid LL-D49194, Meiji Seika ME 2303, menogaril, mithramycin, mitomycin, mitotane, mitoxantrone, SmithKline M-TAG, nemorubicin, neocarzinostatin, neoenactin, neomycin, nisamycin, Nippon Kayaku NK-313, Nippon Kayaku NKT-01, nogalamycin, SR1 International NSC-357704, oxalysine, oxaunomycin, pactamycin, parabactin, paromomycin, peliomycin, peplomycin, pilatin, pirarubicin, plicamycin, porfiromycin, porothramycin, puromycin, pyrindanycin A, Tobishi RA-I, rapamycin, rhizoxin, rodorubicin, sarcomycin, showdomycin, sibanomicin, siwenmycin, Sumitomo SM-5887, Snow Brand SN-706, Snow Brand SN-07, sorangicin-A, sparsomycin, spectinomycin, SS Pharmaceutical SS-21020, SS Pharmaceutical SS-7313B, SS Pharmaceutical SS-9816B, steffimycin B, streptomycin, Taiho 4181-2, talisomycin, Takeda TAN-868A, terpentecin, tetracycline, thrazine, tricrozarin A, Upjohn U-73975, Kyowa Hakko UCN-10028A, valrubicin, Fujisawa WF-3405, Yoshitomi Y-25024 and zorubicin, or a tautomer, prodrug, solvate, or salt thereof.

A sixth family of antineoplastic agents which may be used in combination with compounds of the present invention consists of a miscellaneous family of antineoplastic agents, including tubulin interacting agents, topoisomerase I inhibitors, topoisomerase II inhibitors, and hormonal agents, selected from but not limited to the group consisting of α-carotene, α-difluoromethyl-arginine, acitretin, acodazole, alitretinoin, alstonine, HEXALEN® (altretamine), [1S-[1R*,3R*(E),7R*, [1S-[1R*,3R*(E),7R*,10S*,11R*,12R*,16S*]]-3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione (disclosed in U.S. Pat. No. 6,262,094, which is hereby incorporated by reference), amonafide, amphethinile, amsacrine, antineoplaston A10, antineoplaston A2, antineoplaston A3, antineoplaston A5, antineoplaston AS2-1, Henkel APD, aphidicolin glycinate, arglabin, asparaginase, atrsacrine, avarol, baccharin, batimastat, batracylin, benfluron, benzotript, Ipsen-Beaufour BIM-23015, bisantrene, Bristol-Myers BMY-40481, Vestar 10B, bromofosfamide, 3′-tert-butyl-3′-N-tert-butyloxycarbonyl-4-deacetyl-3′-dephenyl-3′-N-debenzoyl-4-O-methoxycarbonylpaclitaxel, Wellcome BW-502, Wellcome BW-773, caracemide, carmethizole hydrochloride, Ajinomoto CDAF, chlorsulfaquinoxalone, Chemex CHX-2053, Chemex CHX-100, Warner-Lambert CI-921, Warner-Lambert CI-937, Warner-Lambert CI-941, Warner-Lambert CI-958, clanfenur, claviridenone, ICN compound 1259, ICM compound 4711, contracan, Yakult Honsha CPT-11, crisnatol, curaderm, cytochalasin B, cytocytin, Merz D-609, DABIS maleate, dacarbazine, datelliptinium, 4-desacetyl-4-methylcarbonatepaclitaxel, desoxyepothilone A, desoxyepothilone B, didemnin-B, dihaematoporphyrin ether, dihydrolenperone, 10S*,11R*,12R*,16S*]]-7-11-dihydroxy-8,8,10,12,16-pentamethyl-3-[1-methyl-2-(2-methyl-4-thiazolyl)ethenyl]-4-aza-17-oxabicyclo[14.1.0]heptadecane-5,9-dione (disclosed in WO 99/02514), dinaline, Toyo Pharmar DM-341, Toyo Pharmar DM-75, Daiichi Seiyaku DN-9693, TAXOTERE® (docetaxel), docetaxel elliprabin, edelfosine, elliptinium acetate, Tsumura EPMTC, epothilone A, epothilone B, epothilone C, epothilone D, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, ilomastat, irinotecan, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, lurtotecan, Lilly LY-186641, NCI (US) MAP, marimastat, marycin, Merrell Dow MDL-27048, Medco MEDR-340, merbarone, merocyanine derivatives, methylanilinoacridine, C4-methylcarbonatepaclitaxel (disclosed in WO 94/14787), 7-O-methylthiomethylpaclitaxel (disclosed in U.S. Pat. No. 5,646,176, which is hereby incorporated by reference), Molecular Genetics MGI-136, miltefosine, minactivin, mitonafide, mitoquidone mopidamol, motretinide, Zenyaku Kogyo MST-16, N-(retinoyl)amino acids, N-acylated-dehydroalanines, nafazatrom, Taisho NCU-190, nocodazole derivative, Normosang, NCI NSC-145813, NCI NSC-361456, NCI NSC-604782, NCI NSC-95580, ocreotide, Ono ONO-112, oquizanocine, Akzo Org-10172, TAXOL® (paclitaxel) and paclitaxel derivatives and analogues disclosed in U.S. Pat. Nos. 5,569,729; 5,565,478; 5,530,020; 5,527,924; 5,508,447; 5,489,589; 5,488,116; 5,484,809; 5,478,854; 5,478,736; 5,475,120; 5,468,769; 5,461,169; 5,440,057; 5,422,364; 5,411,984; 5,405,972; and 5,296,506, each of which is incorporated by reference in its entirety, pancratistatin, pazelliptine, Warner-Lambert PD-111707, Warner-Lambert PD-115934, Warner-Lambert PD-131141, Pierre Fabre PE-1001, ICRT peptide D, piroxantrone, polyhaematoporphyrin, polypreic acid, Efamol porphyrin, prinomastat, probimane, procarbazine, proglumide, Invitron protease nexin I, Tobishi RA-700, razoxane, Sapporo Breweries RBS, restrictin-P, retelliptine, retinoic acid, Rhone-Poulenc RP-49532, Rhone-Poulenc RP-56976, ricin, rubitecan, a-sarcin, shiga toxin, SmithKline SK&F-104864, Sumitomo SM-108, Kuraray SMANCS, SeaPharm SP-10094, spirocyclopropane derivatives, spirogermanium, SS Pharmaceutical SS-554, stypoldione, Suntory SUN 0237, Suntory SUN 2071, superoxide dismutase, Toyama T-506, Toyama T-680, Teijin TEI-0303, teniposide, thaliblastine, Eastman Kodak TJB-29, tocotrienol, topotecan, Topostin, Teijin TT-82, Kyowa Hakko UCN-01, Kyowa Hakko UCN-1028, ukrain, Eastman Kodak USB-006, vinblastine, vincristine, vindesine, vinestramide, vinorelbine, vintriptol, vinzolidine, and Yamanouchi YM-534, or a tautomer, prodrug, solvate, or salt thereof.

Compounds of the invention may be made by the methods disclosed in U.S. Patent Application Pub. No. 2004/0023999, U.S. Ser. No. 10/947,420, filed Sep. 22, 2004, U.S. Ser. No. 10/947,420, filed Sep. 24, 2003, and U.S. Ser. No. 60/507,079, filed Sep. 29, 2003, each of which is hereby incorporated by reference in its entirety.

Assessment of Biological Properties

Compounds of the invention were evaluated for inhibiting aromatase enzyme using the following aromatase (CYP19) inhibition assay which detects aromatase inhibitors utilizing recombinant human aromatase (baculovirus/insect cell expressed) and the fluorometric substrate dibenzylfluorescein (DBF). Aromatase will metabolize DBF to fluorescein which will give the fluorescent signal. The assay is conducted in 96-well black, Falcon plate (BD Biosciences, Cat. No. 353241) in the total volume of 100 μL per well. 40 μL of the Cofactor/Dilution buffer (1) is added to the plate followed by 10 μL of serially diluted test compounds in duplicates for final concentration of 2×10⁻⁵ to 10⁻⁹ M, or 10 μL of phosphate buffer for positive and background control wells. After 10 minutes of preincubation at 37° C., the reaction is initiated by adding 50 μL of prewarmed Enzyme/Substrate Mix (2) to the test compound wells, and positive control wells (maximum activity of CYP19, which represents 100%). The plate is incubated at 37° C. for 30 minutes. To stop the reaction, 37.5 μL of 2 N NaOH is added to all wells. For background controls, 50 μL of Enzyme/Substrate Mix (2) are added into background control wells. The plate is left at 37° C. incubator for 2 hours to develop adequate signal to background ratio. Fluorescence per well is measured in the fluorescent plate reader. The metabolite concentration is measured using excitation wavelength of 485 nm and emission wavelength of 538 nm. Inhibition of aromatase by test compounds is expressed in percentage relative to positive controls. IC₅₀ values for the test compounds are derived from non-liner curve fitting.
(1) Cofactor/Dilution buffer: 50 mM phosphate buffer, pH=7.4; 1× NADPH regenerating system A (20× stock, GENTEST, Cat. No. 451220); 1× NAPDH regenerating system B (100× stock, GENTEST, Cat. No. 451200) and 0.2 mg/mL control protein (Insect Cell Control Supersomes, GENTEST, Cat. No. 456200)
(2) Enzyme/Substrate Mix: 50 mM phosphate buffer, pH=7.4, 0.02 nmole/mL human aromatase protein (Human CYP19+P450 Reductase Supersomes, GENTEST, Cat. No. 456260); 0.4 μM dibenzylfluorescein (DBF, GENTEST, Cat. No. 451750, prepared at 2 mM concentration in acetonitrile); 0.5 mg/mL control protein (Insect Cell Control Supersomes, GENTEST, Cat. No. 456200).

The following compounds of Formula (IA) showed aromatase inhibition in this assay:
1,1,1-Trifluoro-4-(5-fluoro-2-methoxyphenyl)-4-methyl-2-(1H-pyrrolo[2,3-c]pyridin-2-ylmethyl)pentan-2-ol;
1,1,1-Trifluoro-4-(3-fluorophenyl)-4-methyl-2-(1H-pyrrolo[2,3-c]pyridin-2-ylmethyl)pentan-2-ol;
1,1,1-Trifluoro-4-(4-fluorophenyl)-4-methyl-2-(1H-pyrrolo[2,3-c]pyridin-2-ylmethyl)pentan-2-ol;
4-(2,3-Dihydrobenzofuran-7-yl)-1,1,1-trifluoro-4-methyl-2-(1H-pyrrolo[2,3-c]pyridin-2-ylmethyl)pentan-2-ol;
1,1,1-Trifluoro-4-methyl-4-phenyl-2-(1H-pyrrolo[2,3-c]pyridin-2-ylmethyl)pentan-2-ol;
1,1,1-Trifluoro-4-(4-fluoro-2-methoxyphenyl)-4-methyl-2-(1H-pyrrolo[2,3-c]pyridin-2-ylmethyl)pentan-2-ol;
1,1,1-Trifluoro-4-methyl-4-phenyl-2-(1H-pyrrolo [3,2-c]pyridin-2-ylmethyl)pentan-2-ol;
1,1,1-Trifluoro-4-(4-fluorophenyl)-4-methyl-2-(1H-pyrrolo [3,2-c]pyridin-2-ylmethyl)pentan-2-ol;
4-(2,3-Dihydrobenzofuran-7-yl)-1,1,1-trifluoro-4-methyl-2-(1H-pyrrolo[3,2-c]pyridin-2-ylmethyl)pentan-2-ol;
5-Fluoro-2-[4,4,4-trifluoro-3-hydroxy-1,1-dimethyl-3-(1H-pyrrolo[3,2-c]pyridin-2-ylmethyl)butyl]phenol;
1,1,1-Trifluoro-4-(3-fluorophenyl)-4-methyl-2-(1H-pyrrolo[3,2-c]pyridin-2-ylmethyl)pentan-2-ol;
1,1,1-Trifluoro-4-(5-fluoro-2-methoxyphenyl)-4-methyl-2-(5H-pyrrolo[3,2-d]pyrimidin-6-ylmethyl)pentan-2-ol;
4-(5-Chloro-2,3-dihydrobenzofuran-7-yl)-1,1,1-trifluoro-4-methyl-2-(6-methyl-1H-pyrrolo[3,2-c]pyridin-2-ylmethyl)pentan-2-ol;
1,1,1-Trifluoro-4-(5-fluoro-2-methylphenyl)-4-methyl-2-(5H-pyrrolo[3,2-d]pyrimidin-6-ylmethyl)pentan-2-ol;
1,1,1-Trifluoro-4-methyl-4-(5-methyl-2,3-dihydrobenzofuran-7-yl)-2-(1H-pyrrolo[2,3-c]pyridin-2-ylmethyl)pentan-2-ol;
1,1,1-Trifluoro-4-(5-fluoro-2-methoxyphenyl)-4-methyl-2-thieno[2,3-c]pyridin-2-ylmethylpentan-2-ol;
2-(3-Dimethylaminomethyl-1H-pyrrolo[3,2-c]pyridin-2-ylmethyl)-1,1,1-trifluoro-4-(5-fluoro-2-methoxyphenyl)-4-methylpentan-2-ol; and
1,1,1-Trifluoro-4-(5-fluoro-2-methoxyphenyl)-4-methyl-2-(7H-pyrrolo[2,3-d]pyrimidin-6-ylmethyl)pentan-2-ol.

The following compounds of Formula (IB) showed aromatase inhibition in this assay
2-Cyclopropyl-4-(5-fluoro-2-methoxyphenyl)-4-methyl-1-(1H-pyrrolo[2,3-c]pyridin-2-yl)pentan-2-ol;
2-Cyclopropyl-4-(5-fluoro-2-methylphenyl)-4-methyl-1-(1H-pyrrolo[2,3-c]pyridin-2-yl)pentan-2-ol;
4-(5-Chloro-2,3-dihydrobenzofuran-7-yl)-2-cyclopropyl-4-methyl-1-(1H-pyrrolo[2,3-c]pyridin-2-yl)pentan-2-ol;
2-Cyclopropyl-4-(5-fluoro-2-methylphenyl)-4-methyl-1-(1H-pyrrolo[3,2-c]pyridin-2-yl)pentan-2-ol;
4-(5-Chloro-2,3-dihydrobenzofuran-7-yl)-2-cyclopropyl-4-methyl-1-(1H-pyrrolo[3,2-c]pyridin-2-yl)pentan-2-ol;
5-(5-Fluoro-2-methoxyphenyl)-2,5-dimethyl-3-(1H-pyrrolo[2,3-c]pyridin-2-ylmethyl)hexan-3-ol;
5-(5-Fluoro-2-methoxyphenyl)-2,2,5-trimethyl-3-(1H-pyrrolo[2,3-c]pyridin-2-ylmethyl)hexan-3-ol;
5-(5-Fluoro-2-methylphenyl)-2,5-dimethyl-3-(1H-pyrrolo[2,3-c]pyridin-2-ylmethyl)hexan-3-ol;
5-(5-Chloro-2,3-dihydrobenzofuran-7-yl)-2,5-dimethyl-3-(1H-pyrrolo[2,3-c]pyridin-2-ylmethyl)hexan-3-ol;
1,1-Difluoro-4-(5-fluoro-2-methoxyphenyl)-4-methyl-2-(1H-pyrrolo[3,2-c]pyridin-2-ylmethyl)pentan-2-ol;
5-(5-Fluoro-2-methoxyphenyl)-2,5-dimethyl-3-(1H-pyrrolo[3,2-c]pyridin-2-ylmethyl)hexan-3-ol, and
5-(5-Fluoro-2-methoxyphenyl)-2,2,5-trimethyl-3-(1H-pyrrolo[3,2-c]pyridin-2-ylmethyl)hexan-3-ol.

The invention also provides methods of inhibiting aromatase enzyme in a patient comprising administering to the patient a compound according to the invention. If the purpose of inhibiting the aromatase enzyme in a patient is to treat a disease-state or condition, the administration preferably comprises a therapeutically or pharmaceutically effective amount of a pharmaceutically acceptable compound according to the invention. If the purpose of inhibiting the aromatase enzyme in a patient is for a diagnostic or other purpose (e.g., to determine the patient's suitability for therapy or sensitivity to various sub-therapeutic doses of the compounds according to the invention), the administration preferably comprises an effective amount of a compound according to the invention, that is, the amount necessary to obtain the desired effect or inhibition.

Methods of Therapeutic Use

As pointed out above, the compounds of the invention are useful in inhibiting the aromatase enzyme. In doing so, these compounds have therapeutic use in treating disease-states and conditions mediated by the aromatase enzyme or that would benefit from inhibition of the aromatase enzyme.

As the compounds of the invention inhibit the aromatase enzyme, they are useful in inhibiting estrogen synthesis and treating estrogen dependent conditions such as mammary and endometrial tumors, endometriosis, gynecomastia, etc. They can be used in patients as drugs, particularly in the form of pharmaceutical compositions as set forth below, for the treatment of disease-states and conditions.

Methods of Diagnostic Use

The compounds of the invention may also be used in diagnostic applications and for commercial and other purposes as standards in competitive binding assays. In such uses, the compounds of the invention may be used in the form of the compounds themselves or they may be modified by attaching a radioisotope, luminescence, fluorescent label or the like in order to obtain a radioisotope, luminescence, or fluorescent probe, as would be known by one of skill in the art and as outlined in Handbook of Fluorescent Probes and Research Chemicals, 6th Edition, R. P. Haugland (ed.), Eugene: Molecular Probes, 1996; Fluorescence and Luminescence Probes for Biological Activity, W. T. Mason (ed.), San Diego: Academic Press, 1993; Receptor-Ligand Interaction, A Practical Approach, E. C. Hulme (ed.), Oxford: IRL Press, 1992, each of which is hereby incorporated by reference in their entireties.

General Administration and Pharmaceutical Compositions

When used as pharmaceuticals, the compounds of the invention are typically administered in the form of a pharmaceutical composition. Such compositions can be prepared using procedures well known in the pharmaceutical art and comprise at least one compound of the invention. The compounds of the invention may also be administered alone or in combination with adjuvants that enhance stability of the compounds of the invention, facilitate administration of pharmaceutical compositions containing them in certain embodiments, provide increased dissolution or dispersion, increased inhibitory activity, provide adjunct therapy, and the like. The compounds according to the invention may be used on their own or in conjunction with other active substances according to the invention, optionally also in conjunction with other pharmacologically active substances. In general, the compounds of this invention are administered in a therapeutically or pharmaceutically effective amount, but may be administered in lower amounts for diagnostic or other purposes.

In particular, the compounds of the invention are useful in combination with tamoxifen, other aromatase inhibitors, or other agents used for hormonal therapy. In recent years, endocrine therapy for breast cancer has focused mainly on tamoxifen, a selective estrogen receptor modulator. However, the efficacy of tamoxifen is only partial. In addition, tamoxifen-treated patients may have a higher risk of endometrial cancer. Aromatase inhibitors have been approved as a second-line treatment for estrogen receptor-positive metastatic breast cancer after first-line treatment with tamoxifen. Non-steroidal aromatase inhibitors, anastrazole and letrozole, have also recently been approved as a first-line endocrine treatment for postmenopausal women with estrogen receptor-positive metastatic breast cancer. However estrogen deprivation by aromatase inhibitors in postmenopausal women may have detrimental effects on bone (See Review in Expert Opin. Pharmacother. 2004, 5(2): 307-316 and references cited therein). Hence compounds of this invention may be useful in combination with agents used for treating osteoporosis.

Administration of the compounds of the invention, in pure form or in an appropriate pharmaceutical composition, can be carried out using any of the accepted modes of administration of pharmaceutical compositions. Thus, administration can be, for example, orally, buccally (e.g., sublingually), nasally, parenterally, topically, transdermally, vaginally, or rectally, in the form of solid, semi-solid, lyophilized powder, or liquid dosage forms, such as, for example, tablets, suppositories, pills, soft elastic and hard gelatin capsules, powders, solutions, suspensions, or aerosols, or the like, preferably in unit dosage forms suitable for simple administration of precise dosages. The pharmaceutical compositions will generally include a conventional pharmaceutical carrier or excipient and a compound of the invention as the/an active agent, and, in addition, may include other medicinal agents, pharmaceutical agents, carriers, adjuvants, diluents, vehicles, or combinations thereof. Such pharmaceutically acceptable excipients, carriers, or additives as well as methods of making pharmaceutical compositions for various modes or administration are well-known to those of skill in the art. The state of the art is evidenced, e.g., by Remington: The Science and Practice of Pharmacy, 20th Edition, A. Gennaro (ed.), Lippincott Williams & Wilkins, 2000; Handbook of Pharmaceutical Additives, Michael & Irene Ash (eds.), Gower, 1995; Handbook of Pharmaceutical Excipients, A. H. Kibbe (ed.), American Pharmaceutical Ass'n, 2000; H. C. Ansel and N. G. Popovish, Pharmaceutical Dosage Forms and Drug Delivery Systems, 5th ed., Lea and Febiger, 1990; each of which is incorporated herein by reference in their entireties to better describe the state of the art.

As one of skill in the art would expect, the forms of the compounds of the invention utilized in a particular pharmaceutical formulation will be selected (e.g., salts) that possess suitable physical characteristics (e.g., water solubility) that is required for the formulation to be efficacious.

Pharmaceutical compositions suitable for buccal (sub-lingual) administration include lozenges comprising a compound of the present invention in a flavored base, usually sucrose, and acacia or tragacanth, and pastilles comprising the compound in an inert base such as gelatin and glycerin or sucrose and acacia.

Pharmaceutical compositions suitable for parenteral administration comprise sterile aqueous preparations of a compound of the present invention. These preparations are preferably administered intravenously, although administration can also be effected by means of subcutaneous, intramuscular, or intradermal injection. Injectable pharmaceutical formulations are commonly based upon injectable sterile saline, phosphate-buffered saline, oleaginous suspensions, or other injectable carriers known in the art and are generally rendered sterile and isotonic with the blood. The injectable pharmaceutical formulations may therefore be provided as a sterile injectable solution or suspension in a nontoxic parenterally acceptable diluent or solvent, including 1,3-butanediol, water, Ringer's solution, isotonic sodium chloride solution, fixed oils such as synthetic mono- or diglycerides, fatty acids such as oleic acid, and the like. Such injectable pharmaceutical formulations are formulated according to the known art using suitable dispersing or setting agents and suspending agents. Injectable compositions will generally contain from 0.1 to 5% w/w of a compound of the invention.

Solid dosage forms for oral administration of the compounds include capsules, tablets, pills, powders, and granules. For such oral administration, a pharmaceutically acceptable composition containing a compound(s) of the invention is formed by the incorporation of any of the normally employed excipients, such as, for example, pharmaceutical grades of mannitol, lactose, starch, pregelatinized starch, magnesium stearate, sodium saccharine, talcum, cellulose ether derivatives, glucose, gelatin, sucrose, citrate, propyl gallate, and the like. Such solid pharmaceutical formulations may include formulations, as are well-known in the art, to provide prolonged or sustained delivery of the drug to the gastrointestinal tract by any number of mechanisms, which include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form.

Liquid dosage forms for oral administration of the compounds include emulsions, microemulsions, solutions, suspensions, syrups, and elixirs, optionally containing pharmaceutical adjuvants in a carrier, such as, for example, water, saline, aqueous dextrose, glycerol, ethanol and the like. These compositions can also contain additional adjuvants such as wetting, emulsifying, suspending, sweetening, flavoring, and perfuming agents.

Topical dosage forms of the compounds include ointments, pastes, creams, lotions, gels, powders, solutions, sprays, inhalants, eye ointments, eye or ear drops, impregnated dressings and aerosols, and may contain appropriate conventional additives such as preservatives, solvents to assist drug penetration and emollients in ointments and creams. Topical application may be once or more than once per day depending upon the usual medical considerations. Furthermore, preferred compounds for the present invention can be administered in intranasal form via topical use of suitable intranasal vehicles. The formulations may also contain compatible conventional carriers, such as cream or ointment bases and ethanol or oleyl alcohol for lotions. Such carriers may be present as from about 1% up to about 98% of the formulation, more usually they will form up to about 80% of the formulation.

Transdermal administration is also possible. Pharmaceutical compositions suitable for transdermal administration can be presented as discrete patches adapted to remain in intimate contact with the epidermis of the recipient for a prolonged period of time. To be administered in the form of a transdermal delivery system, the dosage administration will, of course, be continuous rather than intermittent throughout the dosage regimen. Such patches suitably contain a compound of the invention in an optionally buffered, aqueous solution, dissolved and/or dispersed in an adhesive, or dispersed in a polymer. A suitable concentration of the active compound is about 1% to 35%, preferably about 3% to 15%.

For administration by inhalation, the compounds of the invention are conveniently delivered in the form of an aerosol spray from a pump spray device not requiring a propellant gas or from a pressurized pack or a nebulizer with the use of a suitable propellant, e.g., dichlorodifluoromethane, trichlorofluoromethane, dichlorotetrafluoroethane, tetrafluoroethane, heptafluoropropane, carbon dioxide, or other suitable gas. In any case, the aerosol spray dosage unit may be determined by providing a valve to deliver a metered amount so that the resulting metered dose inhaler (MDI) is used to administer the compounds of the invention in a reproducible and controlled way. Such inhaler, nebulizer, or atomizer devices are known in the prior art, for example, in PCT International Publication Nos. WO 97/12687 (particularly FIG. 6 thereof, which is the basis for the commercial RESPIMAT® nebulizer); WO 94/07607; WO 97/12683; and WO 97/20590, to which reference is hereby made and each of which is incorporated herein by reference in their entireties.

Rectal administration can be effected utilizing unit dose suppositories in which the compound is admixed with low-melting water-soluble or insoluble solids such as fats, cocoa butter, glycerinated gelatin, hydrogenated vegetable oils, mixtures of polyethylene glycols of various molecular weights, or fatty acid esters of polyethylene glycols, or the like. The active compound is usually a minor component, often from about 0.05 to 10% by weight, with the remainder being the base component.

In all of the above pharmaceutical compositions, the compounds of the invention are formulated with an acceptable carrier or excipient. The carriers or excipients used must, of course, be acceptable in the sense of being compatible with the other ingredients of the composition and must not be deleterious to the patient. The carrier or excipient can be a solid or a liquid, or both, and is preferably formulated with the compound of the invention as a unit-dose composition, for example, a tablet, which can contain from 0.05% to 95% by weight of the active compound. Such carriers or excipients include inert fillers or diluents, binders, lubricants, disintegrating agents, solution retardants, resorption accelerators, absorption agents, and coloring agents. Suitable binders include starch, gelatin, natural sugars such as glucose or β-lactose, corn sweeteners, natural and synthetic gums such as acacia, tragacanth or sodium alginate, carboxymethylcellulose, polyethylene glycol, waxes, and the like. Lubricants include sodium oleate, sodium stearate, magnesium stearate, sodium benzoate, sodium acetate, sodium chloride, and the like. Disintegrators include starch, methyl cellulose, agar, bentonite, xanthan gum, and the like.

Generally, a therapeutically effective daily dose is from about 0.001 mg to about 15 mg/kg of body weight per day of a compound of the invention; preferably, from about 0.1 mg to about 10 mg/kg of body weight per day; and most preferably, from about 0.1 mg to about 1.5 mg/kg of body weight per day. For example, for administration to a 70 kg person, the dosage range would be from about 0.07 mg to about 1050 mg per day of a compound of the invention, preferably from about 7.0 mg to about 700 mg per day, and most preferably from about 7.0 mg to about 105 mg per day. Some degree of routine dose optimization may be required to determine an optimal dosing level and pattern.

Pharmaceutically acceptable carriers and excipients encompass all the foregoing additives and the like.

Examples of Pharmaceutical Formulations

A. TABLETS

| Component | Amount per tablet (mg) |
|---|---|
| active substance | 100 |
| lactose | 140 |
| corn starch | 240 |
| polyvinylpyrrolidone | 15 |
| magnesium stearate | 5 |
| TOTAL | 500 |

The finely ground active substance, lactose, and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

B. TABLETS

| Component | Amount per tablet (mg) |
|---|---|
| active substance | 80 |
| lactose | 55 |
| corn starch | 190 |
| polyvinylpyrrolidone | 15 |
| magnesium stearate | 2 |
| microcrystalline cellulose | 35 |
| sodium-carboxymethyl starch | 23 |
| TOTAL | 400 |

The finely ground active substance, some of the corn starch, lactose, microcrystalline cellulose, and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

C. COATED TABLETS

| Component | Amount per tablet (mg) |
|---|---|
| active substance | 5 |
| lactose | 30 |
| corn starch | 41.5 |
| polyvinylpyrrolidone | 3 |
| magnesium stearate | 0.5 |
| TOTAL | 90 |

The active substance, corn starch, lactose, and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

D. CAPSULES

| Component | Amount per capsule (mg) |
|---|---|
| active substance | 50 |
| corn starch | 268.5 |
| magnesium stearate | 1.5 |
| TOTAL | 320 |

The substance and corn starch are mixed and moistened with water. The moist mass is screened and dried. The dry granules are screened and mixed with magnesium stearate. The finished mixture is packed into size 1 hard gelatine capsules.

E. AMPOULE SOLUTION

| Component | Amount per ampoule | |
|---|---|---|
| active substance | 50 | mg |
| sodium chloride | 50 | mg |
| water for inj. | 5 | mL |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilized and sealed by fusion. The ampoules contain 5 mg, 25 mg, and 50 mg of active substance.

F. SUPPOSITORIES

| Component | Amount per suppository (mg) |
|---|---|
| active substance | 50 |
| solid fat | 1650 |
| TOTAL | 1700 |

The hard fat is melted. At 40° C., the ground active substance is homogeneously dispersed therein. The mixture is cooled to 38° C. and poured into slightly chilled suppository molds.

G. METERING AEROSOL

| Component | Amount |
|---|---|
| active substance | 0.005 |
| sorbitan trioleate | 0.1 |
| monofluorotrichloromethane and | to 100 |
| difluorodichloromethane (2:3) | |

The suspension is transferred into a conventional aerosol container with a metering valve. Preferably, 50 μL of suspension are delivered per spray. The active substance may also be metered in higher doses if desired (e.g., 0.02% by weight).

| | |
|---|---|
| Component | Amount |

H. POWDER FOR INHALATION

| | | |
|---|---|---|
| active substance | 1.0 | mg |
| lactose monohydrate | to 25 | mg |

I. POWDER FOR INHALATION

| | | |
|---|---|---|
| active substance | 2.0 | mg |
| lactose monohydrate | to 25 | mg |

J. POWDER FOR INHALATION

| | | |
|---|---|---|
| active substance | 1.0 | mg |
| lactose monohydrate | to 5 | mg |

K. POWDER FOR INHALATION

| | | |
|---|---|---|
| active substance | 2.0 | mg |
| lactose monohydrate | to 5 | mg |

In Examples H, I, J, and K, the powder for inhalation is produced in the usual way by mixing the individual ingredients together.

We claim:

1. A compound of Formula (IA) 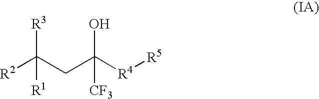
wherein:
R¹ is an aryl or heteroaryl group, each optionally independently substituted with one to three substituent groups,
wherein each substituent group of R¹ is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, C₁-C₅ alkanoyl, aroyl, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, or amino wherein the nitrogen atom is optionally independently mono- or di-substituted by C₁-C₅ alkyl or aryl; or ureido wherein either nitrogen atom is optionally independently substituted with C₁-C₅ alkyl; or C₁-C₅ alkylthio wherein the sulfur atom is optionally oxidized to a sulfoxide or sulfone,
wherein each substituent group of R¹ is optionally independently substituted with one to three substituent groups selected from methyl, methoxy, halogen, hydroxy, oxo, cyano, or amino,
R² and R³ are each independently hydrogen or C₁-C₅ alkyl;
R⁴ is C₁-C₅ alkyl optionally independently substituted with one to three substituent groups,
wherein each substituent group of R⁴ is independently C₁-C₃ alkyl, hydroxy, halogen, amino, or oxo; and
R⁵ is a heteroaryl group optionally independently substituted with one to three substituent groups,
wherein each substituent group of R⁵ is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, or amino wherein the nitrogen atom is optionally independently mono- or di-substituted by C₁-C₅ alkyl; or ureido wherein either nitrogen atom is optionally independently substituted with C₁-C₅ alkyl; or C₁-C₅ alkylthio wherein the sulfur atom is optionally oxidized to a sulfoxide or sulfone,
wherein each substituent group of R⁵ is optionally independently substituted with one to three substituent groups selected from C₁-C₃ alkyl, C₁-C₃ alkoxy, halogen, hydroxy, oxo, cyano, amino, or trifluoromethyl,
or a tautomer, prodrug, solvate, or salt thereof.

2. The compound of Formula (IA) according to claim 1, wherein:
R¹ is phenyl, naphthyl, dihydrobenzofuranyl, benzofuranyl, chromanyl, dihydrobenzothienyl, benzothienyl, benzodioxolanyl, dihydrobenzoxazolyl, benzoxazolyl, benzisoxazolyl, benzpyrazolyl, or benzimidazolyl, each optionally independently substituted with one to three substituent groups,
wherein each substituent group of R¹ is independently C₁-C₃ alkyl, C₂-C₃ alkenyl, C₂-C₃ alkynyl, C₁-C₃ alkoxy, C₂-C₃ alkenyloxy, C₁-C₃ alkanoyl, C₁-C₃ alkoxycarbonyl, C₁-C₃ alkanoyloxy, halogen, hydroxy, carboxy, cyano, trifluoromethyl, trifluoromethoxy, nitro, or C₁-C₃ alkylthio wherein the sulfur atom is optionally oxidized to a sulfoxide or sulfone,
wherein each substituent group of R¹ is optionally independently substituted with a substituent group selected from methyl, methoxy, halogen, hydroxy, oxo, cyano, or amino;
R² and R³ are each independently hydrogen or C₁-C₃ alkyl;
R⁴ is CH₂; and
R⁵ is an indolyl, azaindolyl, diazaindolyl, furanopyridinyl, furanopyrimidinyl, thienopyridinyl, thienopyrimidinyl, oxazolopyridinyl, thiazolopyridinyl, benzimidazolyl, imidazolopyridinyl, quinolinyl, or isoquinolinyl group, each optionally independently substituted with one to three substituent groups,
wherein each substituent group of R⁵ is independently C₁-C₃ alkyl, phenyl, C₁-C₃ alkoxy, methoxycarbonyl, aminocarbonyl, C₁-C₃ alkylaminocarbonyl, C₁-C₃ dialkylaminocarbonyl, heterocyclylcarbonyl, fluoro, chloro, bromo, cyano, trifluoromethyl, or C₁-C₃ alkylthio wherein the sulfur atom is optionally oxidized to a sulfoxide or sulfone,
wherein each substituent group of R⁵ is optionally independently substituted with a substituent group selected from methyl, methoxy, fluoro, chloro, bromo, oxo, trifluoromethyl, or amino wherein the nitrogen atom is optionally independently mono- or di-substituted by C₁-C₅ alkyl or aryl;
or a tautomer, prodrug, solvate, or salt thereof.

3. A compound selected from:
1,1,1-Trifluoro-4-(5-fluoro-2-methoxyphenyl)-4-methyl-2-(1H-pyrrolo[2,3-c]pyridin-2-ylmethyl)pentan-2-ol;
4-Fluoro-2-[4,4,4-trifluoro-3-hydroxy-1,1-dimethyl-3-(1H-pyrrolo[2,3-c]pyridin-2-ylmethyl)butyl]phenol;
1,1,1-Trifluoro-4-(5-fluoro-2-methoxyphenyl)-4-methyl-2-(1H-pyrrolo[3,2-c]pyridin-2-ylmethyl)pentan-2-ol;
4-Fluoro-2-[4,4,4-trifluoro-3-hydroxy-1,1-dimethyl-3-(1H-pyrrolo[3,2-c]pyridin-2-ylmethyl)butyl]phenol;
1,1,1-Trifluoro-4-(3-fluorophenyl)-4-methyl-2-(1H-pyrrolo[2,3-c]pyridin-2-ylmethyl)pentan-2-ol;
1,1,1-Trifluoro-4-(4-fluorophenyl)-4-methyl-2-(1H-pyrrolo[2,3-c]pyridin-2-ylmethyl)pentan-2-ol;
4-(2,3-Dihydrobenzofuran-7-yl)-1,1,1-trifluoro-4-methyl-2-(1H-pyrrolo[2,3-c]pyridin-2-ylmethyl)pentan-2-ol;
1,1,1-Trifluoro-4-methyl-4-phenyl-2-(1H-pyrrolo[2,3-c]pyridin-2-ylmethyl)pentan-2-ol;
1,1,1-Trifluoro-4-(4-fluoro-2-methoxyphenyl)-4-methyl-2-(1H-pyrrolo [2,3-c]pyridin-2-ylmethyl)pentan-2-ol;
1,1,1-Trifluoro-4-methyl-4-phenyl-2-(1H-pyrrolo[3,2-c]pyridin-2-ylmethyl)pentan-2-ol;
1,1,1-Trifluoro-4-(4-fluorophenyl)-4-methyl-2-(1H-pyrrolo[3,2-c]pyridin-2-ylmethyl)pentan-2-ol;
4-(2,3-Dihydrobenzofuran-7-yl)-1,1,1-trifluoro-4-methyl-2-(1H-pyrrolo[3,2-c]pyridin-2-ylmethyl)pentan-2-ol;
1,1,1-Trifluoro-4-(4-fluoro-2-methoxyphenyl)-4-methyl-2-(1H-pyrrolo[3,2-c]pyridin-2-ylmethyl)pentan-2-ol;
5-Fluoro-2-[4,4,4-trifluoro-3-hydroxy-1, 1-dimethyl-3-(1H-pyrrolo[2,3-c]pyridin-2-ylmethyl)butyl]phenol;
1,1,1-Trifluoro-4-(5-fluoro-2-methylphenyl)-4-methyl-2-(1H-pyrrolo[2,3-c]pyridin-2-ylmethyl)pentan-2-ol;
4-Fluoro-2-[4,4,4-trifluoro-3-hydroxy-1,1-dimethyl-3-(3-methyl-1H-pyrrolo[2,3-c]pyridin-2-ylmethyl)butyl]phenol;
5-Fluoro-2-[4,4,4-trifluoro-3-hydroxy-1,1-dimethyl-3-(1H-pyrrolo[3,2-c]pyridin-2-ylmethyl)butyl]phenol;
1,1,1-Trifluoro-4-(3-fluorophenyl)-4-methyl-2-(1H-pyrrolo[3,2-c]pyridin-2-ylmethyl)pentan-2-ol;
1,1,1-Trifluoro-4-(5-fluoro-2,3-dihydrobenzofuran-7-yl)-4-methyl-2-(1H-pyrrolo[2,3-c]pyridin-2-ylmethyl)pentan-2-ol;
4-Fluoro-2-[4,4,4-trifluoro-3-hydroxy-1,1-dimethyl-3-(1H-pyrrolo[2,3-c]-[3-methyl-pyridin]-2-ylmethyl)butyl]phenol;
1,1,1-Trifluoro-4-(5-fluoro-2-methoxyphenyl)-4-methyl-2-(3-methyl-1H-pyrrolo[3,2-c]pyridin-2-ylmethyl)pentan-2-ol;
1,1,1-Trifluoro-4-(5-fluoro-2,3-dihydrobenzofuran-7-yl)-4-methyl-2-(1H-pyrrolo[3,2-c]pyridin-2-ylmethyl)pentan-2-ol;
1,1,1-Trifluoro-4-(5-fluoro-2-methylphenyl)-4-methyl-2-(3-methyl-1H-pyrrolo[2,3-c]pyridin-2-ylmethyl)pentan-2-ol;
1,1,1-Trifluoro-4-(5-fluoro-2-methoxyphenyl)-4-methyl-2-(6-methyl-1H-pyrrolo[3,2-c]pyridin-2-ylmethyl)pentan-2-ol;
1,1,1-Trifluoro-4-(5-fluoro-2-methoxyphenyl)-4-methyl-2-(5H-pyrrolo[3,2-d]pyrimidin-6-ylmethyl)pentan-2-ol;
1,1,1-Trifluoro-4-(5-fluoro-2-methoxyphenyl)₄-methyl-2-(5H-pyrrolo[3,2-c]pyridazin-6-ylmethyl)pentan-2-ol;
1,1,1-Trifluoro-4-(5-fluoro-2-methylphenyl)-4-methyl-2-(1H-pyrrolo[2,3-d]pyridazin-2-ylmethyl)pentan-2-ol;
4-(5-Chloro-2,3-dihydrobenzofuran-7-yl)-1,1,1-trifluoro-4-methyl-2-(6-methyl-1H-pyrrolo[3,2-c]pyridin-2-ylmethyl)pentan-2-ol;
1,1,1-Trifluoro-4-(5-fluoro-2-methylphenyl)-4-methyl-2-(6-methyl-1H-pyrrolo[3,2-c]pyridin-2-ylmethyl)pentan-2-ol;
2-[4-(5-Fluoro-2-methylphenyl)-2-hydroxy-4-methyl-2-trifluoromethylpentyl]-1H-pyrrolo[3,2-b]pyridine-5-carbonitrile;
2-[4-(5-Chloro-2,3-dihydrobenzofuran-7-yl)-2-hydroxy-4-methyl-2-trifluoromethylpentyl]-1H-pyrrolo[3,2-b]pyridine-5-carbonitrile;
1,1,1-Trifluoro-4-(5-fluoro-2-methylphenyl)-4-methyl-2-(5H-pyrrolo[3,2-d]pyrimidin-6-ylmethyl)pentan-2-ol;
4-(5-Chloro-2,3-dihydrobenzofuran-7-yl)-1,1,1-trifluoro-4-methyl-2-(5H-pyrrolo[3,2-d]pyrimidin-6-ylmethyl)pentan-2-ol;
4-(5-Chloro-2,3-dihydrobenzofuran-7-yl)-1,1,1-trifluoro-4-methyl-2-(3-methyl-1H-pyrrolo[2,3-c]pyridin-2-ylmethyl)pentan-2-ol;
1,1,1-Trifluoro-4-(5-fluoro-2-methylphenyl)-4-methyl-2-(5H-pyrrolo[3,2-c]pyridazin-6-ylmethyl)pentan-2-ol;
4-(5-Chloro-2,3-dihydrobenzofuran-7-yl)-1,1,1-trifluoro-4-methyl-2-(5H-pyrrolo[3,2-c]pyridazin-6-ylmethyl)pentan-2-ol;
4-(5-Chloro-2,3-dihydrobenzofuran-7-yl)-1,1,1-trifluoro-4-methyl-2-(1H-pyrrolo[2,3-d]pyridazin-2-ylmethyl)pentan-2-ol;
4-(5-Bromo-2,3-dihydrobenzofuran-7-yl)-1,1,1-trifluoro-4-methyl-2-(1H-pyrrolo[2,3-c]pyridin-2-ylmethyl)pentan-2-ol;
1,1,1-Trifluoro-4-methyl-4-(5-methyl-2,3-dihydrobenzofuran-7-yl)-2-(1H-pyrrolo[2,3-c]pyridin-2-ylmethyl)pentan-2-ol;
4-(5-Chloro-2,3-dihydrobenzofuran-7-yl)-1,1,1-trifluoro-4-methyl-2-(1H-pyrrolo[2,3-c]pyridin-2-ylmethyl)pentan-2-ol;
1,1,1-Trifluoro-4-(5-fluoro-2-methoxyphenyl)-4-methyl-2-(6-oxy-1H-pyrrolo[2,3-c]pyridin-2-ylmethyl)pentan-2-ol;
1,1,1-Trifluoro-4-(5-fluoro-2-methoxyphenyl)-4-methyl-2-thieno[2,3-c]pyridin-2-ylmethylpentan-2-ol;
4-(5-Chloro-2,3-dihydrobenzofuran-7-yl)-2,4-dimethyl-1-thieno[2,3-c]pyridin-2-ylpentan-2-ol;
4-(5-Fluoro-2-methylphenyl)-2,4-dimethyl-1-thieno[2,3-c]pyridin-2-ylpentan-2-ol;
1,1,1-Trifluoro-4-(5-fluoro-2-methoxyphenyl)-2-furo[2,3-c]pyridin-2-ylmethyl-4-methylpentan-2-ol;
4-(5-Chloro-2,3-dihydrobenzofuran-7-yl)-1-furo[2,3-c]pyridin-2-yl-2,4-dimethylpentan-2-ol;
4-(5-Fluoro-2-methylphenyl)-1-furo[2,3-c]pyridin-2-yl-2,4-dimethylpentan-2-ol;
1,1,1-Trifluoro-4-(5-fluoro-2-methylphenyl)-4-methyl-2-(1H-pyrrolo[3,2-c]pyridin-2-ylmethyl)pentan-2-ol;
1,1,1-Trifluoro-4-methyl-4-(5-methyl-2,3-dihydrobenzofuran-7-yl)-2-(1H-pyrrolo[3,2-c]pyridin-2-ylmethyl)pentan-2-ol;
4-(5-Chloro-2,3-dihydrobenzofuran-7-yl)-1,1,1-trifluoro-4-methyl-2-(1H-pyrrolo[3,2-c]pyridin-2-ylmethyl)pentan-2-ol;
4-(5-Bromo-2,3-dihydrobenzofuran-7-yl)-1,1,1-trifluoro-4-methyl-2-(1H-pyrrolo[3,2-c]pyridin-2-ylmethyl)pentan-2-ol;
2-(3-Dimethylaminomethyl-1H-pyrrolo[3,2-c]pyridin-2-ylmethyl)-1,1,1-trifluoro-4-(5-fluoro-2-methoxyphenyl)-4-methylpentan-2-ol;
1,1,1-Trifluoro-4-(5-fluoro-2-methoxyphenyl)-2-furo[3,2-c]pyridin-2-ylmethyl-4-methylpentan-2-ol;
1,1,1-Trifluoro-4-(5-fluoro-2-methoxyphenyl)-4-methyl-2-thieno[3,2-c]pyridin-2-ylmethylpentan-2-ol;
4-(5-Chloro-2,3-dihydrobenzofuran-7-yl)-1,1,1-trifluoro-4-methyl-2-thieno[3,2-c]pyridin-2-ylmethylpentan-2-ol;
1,1,1-Trifluoro-4-(5-fluoro-2-methylphenyl)-4-methyl-2-thieno[3,2-c]pyridin-2-ylmethylpentan-2-ol;
4-Fluoro-2-(4,4,4-trifluoro-3-hydroxy-1, 1-dimethyl-3-thieno[3,2-c]pyridin-2-ylmethylbutyl)phenol;
4-Fluoro-2-(4,4,4-trifluoro-3-furo[3,2-c]pyridin-2-ylmethyl-3-hydroxy-1,1-dimethylbutyl)phenol;
2-(3-Bromo-1H-indol-2-ylmethyl)-1,1,1-trifluoro-4-(3-fluorophenyl)-4-methylpentan-2-ol; and
1,1,1-Trifluoro-4-(5-fluoro-2-methoxyphenyl)-4-methyl-2-(7H-pyrrolo[2,3-d]pyrimidin-6-ylmethyl)pentan-2-ol,
or a tautomer, prodrug, solvate, or salt thereof.

4. A pharmaceutical composition comprising a pharmaceutically effective amount of a compound according to one of claims 1 to 3, or a tautomer, prodrug, solvate, or salt thereof, and a pharmaceutically acceptable excipient or carrier.

5. A pharmaceutical composition comprising a pharmaceutically effective amount of:
(a) a compound according to one of claims 1 to 3, or a tautomer, prodrug, solvate, or salt thereof, and a pharmaceutically acceptable excipient or carrier; and
(b) an additional therapeutic agent.

6. The pharmaceutical composition according to claim 5, wherein the additional therapeutic agent is a selective estrogen-receptor modulator, aromatase inhibitor, biologic response modifier, hormone therapy agent, or chemotherapy agent.

7. A method of treating an estrogen receptor-mediated disorder in a patient in need of such treatment, the method comprising administering to the patient a pharmaceutically effective amount of a pharmaceutically acceptable compound according to one of claims 1 to 3, or a tautomer, prodrug, solvate, or salt thereof.

8. A compound of Formula (IB) 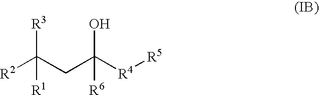
wherein:
R¹ is an aryl or heteroaryl group, each optionally independently substituted with one to three substituent groups,
wherein each substituent group of R¹ is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, C₁-C₅ alkanoyl, aroyl, aminocarbonyl, C₁-C₅ alkylaminocarbonyl, C₁-C₅ dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, aminosulfonyl, C₁-C₅ alkylaminosulfonyl, C₁-C₅ dialkylaminosulfonyl, halogen, hydroxy, carboxy, cyano, trifluoromethyl, trifluoromethoxy, nitro, or amino wherein the nitrogen atom is optionally independently mono- or di-substituted by C₁-C₅ alkyl or aryl; or ureido wherein either nitrogen atom is optionally independently substituted with C₁-C₅ alkyl; or C₁-C₅ alkylthio wherein the sulfur atom is optionally oxidized to a sulfoxide or sulfone,
wherein each substituent group of R¹ is optionally independently substituted with one to three substituent groups selected from methyl, methoxy, halogen, hydroxy, oxo, cyano, or amino,
R² and R³ are each independently C₁-C₅ alkyl;
R⁴ is C₁-C₅ alkyl optionally independently substituted with one to three substituent groups,
wherein each substituent group of R⁴ is independently C₁-C₃ alkyl, hydroxy, halogen, or oxo;
R⁵ is a heteroaryl group optionally independently substituted with one to three substituent groups,
wherein each substituent group of R⁵ is independently C₁-C₅ alkyl, C₂-C₅ alkenyl, C₂-C₅ alkynyl, C₃-C₈ cycloalkyl, heterocyclyl, aryl, heteroaryl, C₁-C₅ alkoxy, C₂-C₅ alkenyloxy, C₂-C₅ alkynyloxy, aryloxy, acyl, C₁-C₅ alkoxycarbonyl, C₁-C₅ alkanoyloxy, aminocarbonyl, alkylaminocarbonyl, dialkylaminocarbonyl, aminocarbonyloxy, C₁-C₅ alkylaminocarbonyloxy, C₁-C₅ dialkylaminocarbonyloxy, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, trifluoromethyl, trifluoromethoxy, trifluoromethylthio, nitro, or amino wherein the nitrogen atom is optionally independently mono- or di-substituted by C₁-C₅ alkyl; or ureido wherein either nitrogen atom is optionally independently substituted with C₁-C₅ alkyl; or C₁-C₅ alkylthio wherein the sulfur atom is optionally oxidized to a sulfoxide or sulfone,
wherein each substituent group of R⁵ is optionally independently substituted with one to three substituent groups selected from C₁-C₃ alkyl, C₁-C₃ alkoxy, halogen, hydroxy, oxo, cyano, amino, or trifluoromethyl; and
R⁶ is C₁-C₈ alkyl, C₂-C₈ alkenyl, C₂-C₈ alkynyl, carbocycle, heterocyclyl, aryl, heteroaryl, carbocycle-C₁-C₈ alkyl, aryl-C₁-C₈ alkyl, aryl-C₁-C₈ haloalkyl, heterocyclyl-C₁-C₈ alkyl, heteroaryl-C₁-C₈ alkyl, carbocycle-C₂-C₈ alkenyl, aryl-C₂-C₈ alkenyl, heterocyclyl-C₂-C₈ alkenyl, or heteroaryl-C₂-C₈ alkenyl, each optionally independently substituted with one to three substituent groups,
wherein each substituent group of R⁶ is independently C₁-C₅ alkyl, C₂-C₅ alkenyl, C₂-C₅ alkynyl, C₃-C₈ cycloalkyl, phenyl, C₁-C₅ alkoxy, phenoxy, C₁-C₅ alkanoyl, aroyl, C₁-C₅ alkoxycarbonyl, C₁-C₅ alkanoyloxy, aminocarbonyloxy, C₁-C₅ alkylaminocarbonyloxy, C₁-C₅ dialkylaminocarbonyloxy, aminocarbonyl, C₁-C₅ alkylaminocarbonyl, C₁-C₅ dialkylaminocarbonyl, C₁-C₅ alkanoylamino, C₁-C₅ alkoxycarbonylamino, C₁-C₅ alkylsulfonylamino, C₁-C₅ alkylaminosulfonyl, C₁-C₅ dialkylaminosulfonyl, halogen, hydroxy, carboxy, cyano, oxo, trifluoromethyl, nitro, amino wherein the nitrogen atom is optionally independently mono- or di-substituted by C₁-C₅ alkyl; or ureido wherein either nitrogen atom is optionally independently substituted with C₁-C₅ alkyl; or C₁-C₅ alkylthio wherein the sulfur atom is optionally oxidized to a sulfoxide or sulfone,
wherein R⁶ cannot be trifluoromethyl,
or a tautomer, prodrug, solvate, or salt thereof.

9. The compound of Formula (IB) according to claim 8, wherein:
R¹ is phenyl, naphthyl, dihydrobenzofuranyl, benzofuranyl, chromanyl, dihydrobenzothienyl, benzothienyl, benzodioxolanyl, benzoxazolyl, benzisoxazolyl, benzpyrazolyl, or benzimidazolyl, each optionally independently substituted with one to three substituent groups,
wherein each substituent group of R¹ is independently C₁-C₃ alkyl, C₂-C₃ alkenyl, C₂-C₃ alkynyl, C₁-C₃ alkoxy, C₂-C₃ alkenyloxy, C₁-C₃ alkanoyl, C₁-C₃ alkoxycarbonyl, C₁-C₃ alkanoyloxy, halogen, hydroxy, carboxy, cyano, trifluoromethyl, nitro, or C₁-C₃ alkylthio wherein the sulfur atom is optionally oxidized to a sulfoxide or sulfone,
wherein each substituent group of R¹ is optionally independently substituted with a substituent group selected from methyl, methoxy, halogen, hydroxy, oxo, cyano, or amino;
R² and R³ are each independently C₁-C₃ alkyl;
R⁴ is CH₂;
R⁵ is an indolyl, azaindolyl, diazaindolyl, furanopyridinyl, furanopyrimidinyl, thienopyridinyl, thienopyrimidinyl, oxazolopyridinyl, thiazolopyridinyl, benzimidazolyl, imidazolopyridinyl, quinolinyl, or isoquinolinyl group, each optionally independently substituted with one to three substituent groups,
wherein each substituent group of R⁵ is independently C₁-C₃ alkyl, phenyl, C₁-C₃ alkoxy, methoxycarbonyl, aminocarbonyl, C₁-C₃ alkylaminocarbonyl, C₁-C₃ dialkylaminocarbonyl, heterocyclylcarbonyl, fluoro, chloro, bromo, cyano, trifluoromethyl, or C₁-C₃ alkylthio wherein the sulfur atom is optionally oxidized to a sulfoxide or sulfone,
wherein each substituent group of R⁵ is optionally independently substituted with a substituent group selected from methyl, methoxy, fluoro, chloro, bromo, trifluoromethyl, or amino wherein the nitrogen atom is optionally independently mono- or di-substituted by C₁-C₅ alkyl or aryl or trifluoromethyl; and
R⁶ is C₁-C₅ alkyl, C₂-C₅ alkenyl, C₃-C₆ cycloalkyl, phenyl, C₃-C₆ cycloalkyl-C₁-C₃ alkyl, phenyl-C₁-C₃ alkyl, phenyl-C₁-C₃ haloalkyl, C₃-C₆ cycloalkyl-C₂-C₃ alkenyl, phenyl-C₂-C₃ alkenyl, each optionally independently substituted with one to three substituent groups,
wherein each substituent group of R⁶ is independently C₁-C₃ alkyl, C₂-C₃ alkenyl, C₂-C₃ alkynyl, C₁-C₃ alkoxy, aminocarbonyl, C₁-C₃ alkylaminocarbonyl, C₁-C₃ dialkylaminocarbonyl, halogen, hydroxy, carboxy, cyano, trifluoromethyl, nitro, or C₁-C₃ alkylthio wherein the sulfur atom is optionally oxidized to a sulfoxide or sulfone,
or a tautomer, prodrug, solvate, or salt thereof.

10. A compound selected from:
2-Cyclopropyl-4-(5-fluoro-2-methoxyphenyl)-4-methyl-1-(1H-pyrrolo[2,3-c]pyridin-2-yl)pentan-2-ol;
4-(5-Bromo-2,3-dihydrobenzofuran-7-yl)-2-cyclopropyl-4-methyl-1-(1H-pyrrolo[2,3-c]pyridin-2-yl)pentan-2-ol;
2-Cyclopropyl-4-(5-fluoro-2-methoxyphenyl)-4-methyl-1-(1H-pyrrolo[3,2-c]pyridin-2-yl)pentan-2-ol;
4-(5-Fluoro-2-methoxyphenyl)-2-hydroxy-4-methyl-2-(1H-pyrrolo[2,3-c]pyridin-2-ylmethyl)pentanoic acid;
4-(5-Fluoro-2-methoxyphenyl)-2-hydroxy-4-methyl-2-(1H-pyrrolo[2,3-c]pyridin-2-ylmethyl)pentanoic acid methyl ester;
2-Cyclopropyl-4-(5-fluoro-2-methylphenyl)-4-methyl-1-(1H-pyrrolo[2,3-c]pyridin-2-yl)pentan-2-ol;
4-(5-Chloro-2,3-dihydrobenzofuran-7-yl)-2-cyclopropyl-4-methyl-1-(1H-pyrrolo[2,3-c]pyridin-2-yl)pentan-2-ol;
2-Cyclopropyl-4-(5-fluoro-2-methylphenyl)-4-methyl-1-(1H-pyrrolo[3,2-c]pyridin-2-yl)pentan-2-ol;
4-(5-Chloro-2,3-dihydrobenzofuran-7-yl)-2-cyclopropyl-4-methyl-1-(1H-pyrrolo[3,2-c]pyridin-2-yl)pentan-2-ol;
4-(5-Fluoro-2-methoxyphenyl)-2,4-dimethyl-1-(1H-pyrrolo[2,3-c]pyridin-2-yl)pentan-2-ol;
5-(5-Fluoro-2-methoxyphenyl)-2,5-dimethyl-3-(1H-pyrrolo[2,3-c]pyridin-2-ylmethyl)hexan-3-ol;
5-(5-Fluoro-2-methoxyphenyl)-2,2,5-trimethyl-3-(1H-pyrrolo[2,3-c]pyridin-2-ylmethyl)hexan-3-ol;
2-Cyclohexyl-4-(5-fluoro-2-methoxyphenyl)-4-methyl-1-(1H-pyrrolo[2,3-c]pyridin-2-yl)pentan-2-ol;
2-Cyclopentyl-4-(5-fluoro-2-methoxyphenyl)-4-methyl-1-(1H-pyrrolo[2,3-c]pyridin-2-yl)pentan-2-ol;
5-(5-Fluoro-2-methoxyphenyl)-5-methyl-3-(1H-pyrrolo[2,3-c]pyridin-2-ylmethyl)hexan-3-ol;
2-(5-Fluoro-2-methoxyphenyl)-2,6-dimethyl-4-(1H-pyrrolo[2,3-c]pyridin-2-ylmethyl)heptan-4-ol;
2-(5-Fluoro-2-methoxyphenyl)-2,5,5-trimethyl-4-(1H-pyrrolo[2,3-c]pyridin-2-ylmethyl)heptan-4-ol;
1,1-Difluoro-4-(5-fluoro-2-methoxyphenyl)-4-methyl-2-(1H-pyrrolo[2,3-c]pyridin-2-ylmethyl)pentan-2-ol;
1-Cyclohexyl-4-(5-fluoro-2-methoxyphenyl)-4-methyl-2-(1H-pyrrolo[2,3-c]pyridin-2-ylmethyl)pentan-2-ol;
5-(5-Fluoro-2-methylphenyl)-2,5-dimethyl-3-(1H-pyrrolo [2,3-c]pyridin-2-ylmethyl)hexan-3-ol;
5-(5-Fluoro-2-methylphenyl)-2,2,5-trimethyl-3-(1H-pyrrolo[2,3-c]pyridin-2-ylmethyl)hexan-3-ol;
5-(5-Chloro-2,3-dihydrobenzofuran-7-yl)-2,5-dimethyl-3-(1H-pyrrolo[2,3-c]pyridin-2-ylmethyl)hexan-3-ol;
2-Cyclobutyl-4-(5-fluoro-2-methoxyphenyl)-4-methyl-1-(1H-pyrrolo[2,3-c]pyridin-2-yl)pentan-2-ol;
2-(5-Fluoro-2-methoxyphenyl)-2,6,6-trimethyl-4-(1H-pyrrolo[2,3-c]pyridin-2-ylmethyl)heptan-4-ol;
5-(5-Fluoro-2-methoxyphenyl)-5-methyl-3-(1H-pyrrolo[2,3-c]pyridin-2-ylmethyl)hex-1-en-3-ol;
5-(5-Fluoro-2-methoxyphenyl)-5-methyl-3-(1H-pyrrolo[2,3-c]pyridin-2-ylmethyl)hex-1-yn-3-ol;
1-Fluoro-4-(5-fluoro-2-methoxyphenyl)-4-methyl-2-(1H-pyrrolo [2,3-c]pyridin-2-ylmethyl)pentan-2-ol;
2,2-Difluoro-5-(5-fluoro-2-methoxyphenyl)-5-methyl-3-(1H-pyrrolo[2,3-c]pyridin-2-ylmethyl)hexan-3-ol;
2-Fluoro-5-(5-fluoro-2-methoxyphenyl)-2,5-dimethyl-3-(1H-pyrrolo [2,3-c]pyridin-2-ylmethyl)hexan-3-ol;
2-Fluoro-5-(5-fluoro-2-methoxyphenyl)-5-methyl-3-(1H-pyrrolo[2,3-c]pyridin-2-ylmethyl)hexan-3-ol;
5-(5-Fluoro-2-methoxyphenyl)-2,5-dimethyl-3-(1H-pyrrolo[2,3-c]pyridin-2-ylmethyl)hex-1-en-3-ol;
1,1,1-Trifluoro-5-(5-fluoro-2-methoxyphenyl)-5-methyl-3-(1H-pyrrolo[2,3-c]pyridin-2-ylmethyl)hexan-3-ol;
4-(5-Fluoro-2-methoxyphenyl)-4-methyl-2-phenyl-1-(1H-pyrrolo[2,3-c]pyridin-2-yl)pentan-2-ol;
5-(5-Chloro-2,3-dihydrobenzofuran-7-yl)-2,2,5-trimethyl-3-(1H-pyrrolo[2,3-c]pyridin-2-ylmethyl)hexan-3-ol;
5-(5-Fluoro-2-methylphenyl)-2,2,5-trimethyl-3-thieno[2,3-c]pyridin-2-ylmethylhexan-3-ol;
1,1-Difluoro-4-(5-fluoro-2-methoxyphenyl)-4-methyl-2-(1H-pyrrolo[3,2-c]pyridin-2-ylmethyl)pentan-2-ol;
5-(5-Fluoro-2-methoxyphenyl)-2,5-dimethyl-3-(1H-pyrrolo[3,2-c]pyridin-2-ylmethyl)hexan-3-ol;
5-(5-Fluoro-2-methoxyphenyl)-2,2,5-trimethyl-3-(1H-pyrrolo[3,2-c]pyridin-2-ylmethyl)hexan-3-ol;
2-(1-Fluorocyclopropyl)-4-(5-fluoro-2-methoxyphenyl)-4-methyl-1-(1H-pyrrolo[2,3-c]pyridin-2-yl)pentan-2-ol;
2-(1-Fluorocyclopropyl)-4-(4-fluorophenyl)-4-methyl-1-quinolin-4-ylpentan-2-ol,
or a tautomer, prodrug, solvate, or salt thereof.

11. A pharmaceutical composition comprising a pharmaceutically effective amount of a compound according to one of claims 8 to 10, or a tautomer, prodrug, solvate, or salt thereof, and a pharmaceutically acceptable excipient or carrier.

12. A pharmaceutical composition comprising a pharmaceutically effective amount of:
(a) a compound according to one of claims 8 to 10, or a tautomer, prodrug, solvate, or salt thereof, and a pharmaceutically acceptable excipient or carrier; and
(b) an additional therapeutic agent.

13. The pharmaceutical composition according to claim 12, wherein the additional therapeutic agent is a selective estrogen-receptor modulator, aromatase inhibitor, biologic response modifier, hormone therapy agent, or chemotherapy agent.

14. A method of treating an estrogen receptor-mediated disorder in a patient in need of such treatment, the method comprising administering to the patient a pharmaceutically effective amount of a pharmaceutically acceptable compound according to one of claims 8 to 10, or a tautomer, prodrug, solvate, or salt thereof.
